(19) United States
(12) Patent Application Publication
(10) Pub. No.: US 2026/0354905 A1
(43) Pub. Date: Nov. 12, 2026

(54) LOCALIZATION DEVICE AND METHOD

(71) Applicant: Deutsches Krebsforschungszentrum Stiftung des öffentlichen Rechts, Heidelberg (DE)

(72) Inventors: Tian Qiu, Stuttgart (DE); Felix Fischer, Stuttgart (DE); Christian Gletter, Stuttgart (DE)

(21) Appl. No.: 19/103,063

(22) PCT Filed: Aug. 10, 2023
(86) PCT No.: PCT/EP2023/072144

§ 371 Date: Feb. 11, 2025

(30) Foreign Application Priority Data
Aug. 12, 2022 (DE) 10 2022 120 495.1

(51) Int. Cl. A61B 34/20; A61B 90/00

(52) U.S. Cl. CPC A61B 34/20; A61B 90/39; A61B 2034/2051; A61B 2090/3958

(57) ABSTRACT

A method for determining a position and an orientation of a localization device, the localization device having a magnet attached to an oscillating element. The method comprises exciting the magnet using one of an external force or torque, resulting in a complex oscillatory motion of the magnet. The said complex motion comprises translation and rotation of the magnetic moment of the magnet about a rotational axis and the rotational axis is located at an offset distance relative to the center of the magnetic moment of the magnet. The method comprises sensing the magnetic field generated by the magnet using at least one sensor. The method comprises determining the position and the orientation of the localization device from the sensing.

### CROSS-REFERENCE TO RELATED APPLICATIONS

This application claims priority to German Patent Application 10 2022 120 495.2, filed on 12 Aug. 2022. The entire disclosure of German Patent Application 10 2022 120 495.2 is hereby incorporated herein by reference.

### FIELD OF THE INVENTION

The invention relates to a system and method for determining the position and orientation of a localization device.

### BACKGROUND OF THE INVENTION

Localization and tracking of medical robots or tools are required for the success of in vivo surgical procedures and diagnostics. In particular, methods and devices which can be used without particular care for noise interference or signal absorption from biological tissue are at the focus of current research.

Wireless biomedical devices such as endoscopy capsules and tissue markers require reliable localization and tracking with trackers with high spatial and temporal resolution in order to increase the success of medical procedures or diagnostics.

A number of localization methods have been developed over the years. However, they suffer from a number of disadvantages. For example, harmful radiation from X-Ray or computed tomography (CT) or easily disturbed magnetic resonance imaging (MRI) are well-established localization techniques for medical robots and tools. However, they cannot be used during a typical surgical operation, as they require special rooms, large machinery, and careful preparation. Millimeter-sized electromagnetic (EM) trackers can be embedded in, for example, diseased tissue under local imaging guidance and tracked in 3D for more efficient radiation therapies. Other trackers, for example trackers with embedded sensors, can provide high spatial resolution, however they are typically too large for a feasible application in the human body.

From millimeter- to nanometer-scale, robots have gained significant attention over the last decades thanks to increasingly sophisticated fabrication techniques, inclusion of multi-functional materials and design approaches. These robots have been used in medical applications. Current research aims for multi-functionality, miniaturization, device autonomy, energy efficiency and biocompatibility. While highly developed macroscopic devices such as endoscopes and catheters are in clinical everyday use, wireless millimeter to nanometer scale devices are treated cautiously due to insufficient position feedback and inaccurate control.

Ultrasound (US) systems have also been successfully demonstrated for localization of small devices. Intracorporeal applications of such US systems suffer from distortion due to inhomogeneous wave propagation properties of the different biological materials (e.g., bones, organs, muscles), do not provide full spatial information and US sources are difficult to implement into millimeter scale devices.

Electromagnetic (EM) waves at low frequencies and radio frequencies (RF) between several kHz to GHz have very low attenuation in biological materials and exhibit no harmful effects which makes them suitable for use as trackers in human applications. Spatial resolution below 2 mm and a temporal resolution of 10 Hz has been achieved with multiple embedded coils with a relatively large size of 8 mm length each. Using a highly sophisticated chip design, an integrated sensor circuit has been designed to measure 3D magnetic field gradients on-board with a sub-millimeter resolution at 10 Hz. However, RF methods, despite their large penetration depth and high accuracy, are susceptible to magnetic objects in proximity to the tracker. Currently, permanent magnets embedded in the trackers have to be comparably large (>6 mm³) in order to generate a measurable field in deep biological tissue but are ideal for the purpose of magnetic actuation.

Widely used in the medical field, magnetic resonance imaging (MRI) is a localization technique which allows in vivo scanning of tissue in 3D. However, MRI suffers from low temporal resolution above 1 Hz at also limited spatial resolution, which makes it unsuitable for tracking of sub-millimetric markers.

Plotkin and Paperno studied sensors inside a magnetic field in “3-D Magnetic Tracking of a Single Subminiature Coil with a Large 2-D Array of Uniaxial Transmitters”, IEEE Transactions on Magnetics, September 2003, Vol. 5, p. 3295. The authors reported development of a magnetic sensor measuring a magnetic field generated by external electromagnetic coils. The magnetic device requires a wired connection for energy supply or energy reception and is easily disturbed by medical devices and ferromagnetic objects.

Atuegwu and Galloway described a magnetic tracking system in their study “Volumetric characterization of the Aurora magnetic tracker system for image-guided transorbital endoscopic procedures” in Physics in Medicine & Biology, 2008, Vol. 53, p. 4355-4368. The magnetic tracking system is used in an image-guided procedure. The magnetic tracking system comprises a field generator and a coil sensor. The field generator comprises coils that generate an electromagnetic field. The coil sensor measures the voltage induced by the generated magnetic field in a coil. The induced voltage is then used to calculate the position and orientation of the coil sensor. The coil sensor needs a wired connection for energy supply. The coil is easily disturbed by medical devices and ferromagnetic objects.

Sharma et al. in “Wireless 3D Surgical Navigation and Tracking System With 100 μm Accuracy Using Magnetic-Field Gradient-Based Localization” IEEE Transactions on Medical Imaging, August 2021, Vol. 40, No. 8, p. 2066-2078, disclose a wireless tracking system using magnetic field gradients. The tracking system is actuated in the human body during surgeries and diagnostic procedures. The tracking system comprises a micro-chip, a magnetic sensor, and an inductor coil. The tracking system is sensitive to surrounding magnetic fields and the implantable micro-chip is large.

Sensed magnetic field generators are described by Fernandez et al. in “High-Accuracy Wireless 6DOF Magnetic Tracking System Based on FEM Modeling,” IEEE International Conference on Electronics, Circuits and Systems (ICECS), 2018 25th, p. 413-416. The authors disclose a magnetic tracking system based on a simplified position estimation algorithm. The magnetic tracking system comprises a fixed field generator module for generating a known static magnetic field. The magnetic tracking system further comprises a movable receiver module for sensing a generated field, for processing the value of the coordinates of the generated magnetic field and for transmitting the estimated position and orientation of marker points. The magnetic tracking system is based on a wired method.

Chinese Patent Application No. CN102274024A discloses a microprocessor-based dual magnet bar rotation search, positioning and tracking system comprising two magnet bars, a magnet bar excitation circuit, a rotating device, a magneto resistive sensor, a signal conditioning circuit, an ADC sampling circuit, and a control processing unit. The magnet bars comprise electromagnetic coils. The magnet bar excitation circuit generates a magnetic field exciting the electromagnetic coils. The rotating device comprises a horizontal rotation stepper motor and a vertical rotation stepper motor, and the two groups of rotation devices control the two magnetic bars to rotate freely in the horizontal and vertical directions, respectively.

A number of studies have been conducted on systems comprising permanent magnets for determining a position and an orientation of a localization device. Son et al. discloses in “A 5-D Localization Method for a Magnetically Manipulated Untethered Robot Using a 2-D Array of Hall-Effect Sensors”, EEE/ASME Transactions on Mechatronics, 2016, Vol. 21, No. 2, p. 708-716, a system based on localizing a tracker by sensing a static magnetic field. The system comprises a large permanent magnet generating a magnetic field. A magnet used for actuation comprises three box-shaped orthogonal coils and a soft iron core. The system further comprises a sensor array board comprising sixty-four Hall-effect sensors measuring the magnetic field in the direction perpendicular to the array.

Nicolae et al. describe in “Evaluation of a Ferromagnetic Marker Technology for Intraoperative Localization of Nonpalpable Breast Lesions,” American Journal of Roentgenology, April 2019, p. 727-733, a handheld probe that can detect the position and distance of a magnetic marker. A method for localizing the magnetic marker comprises implanting a magnetic marker in a breast lesion and sensing the persistent magnetic field with the handheld probe.

Methods based on passive transponders are also used for determining a position and an orientation of a localization device. Passive transponders comprise an embedded electromagnetic (EM) coil which operates at radio frequency. The method comprises recording the magnitude of the magnetic moment of electromagnetic circuits and calculating the phase difference of the emitted signals and received signals. Willoughby et al. disclose the use of passive transponders in “Target localization and real-time tracking using the calypso 4d localization system in patients with localized prostate cancer” Int. J. Radiation Oncology Biol. Phys., 2006, Vol. 65, No. 2, p. 528-534, as well as Hekimian-Williams et al. in “Accurate Localization of RFID Tags Using Phase Difference” IEEE RFID, 2010, p. 89-96.

Mechanically resonant magnetic structures are known as magnetic field sensors. Japanese Patent Application No. JP2005201775 discloses a magnetic field sensor comprising a substrate onto which a vibrating plate is formed. The sensor further comprises a ferromagnetic thin film on the vibrating plate. JP2005201775 discloses a magnetic field sensor comprising an excitation device for exciting the vibrating plate and a detection device for detecting the resonance frequency of the vibrating plate, which is dependent on an external magnetic field.

U.S. Pat. No. 8,519,810 B2 discloses a system for magnetic proximity determination. The system comprises a substrate and contacts supported by the substrate. A moveable element comprising two distinguishing ends is attached to the substrate. A first permanent magnet is disposed near the first end of the movable element to produce a first magnetic attraction force and a first torque on the moveable element. A second movable magnet is disposed near the second end of the movable element to produce a second magnetic attraction force and a second torque about said rotational axis.

European Patent Application No. EP 3 583 896 A1 discloses a method for tracking a marker device or a localization device. The marker device comprises a rotationally oscillating magnetic object and a restoring torque unit forcing the magnetic object back into an equilibrium position if an external magnetic field has rotated the magnetic object out of its equilibrium position. The disclosed method comprises a step of generating a magnetic field resulting in a rotational oscillation of the magnetic object out of its equilibrium position. The rotational oscillation of the magnetic object generates induction signals. The induction signals are sensed and thereby enable the determination of the position and the orientation of the marker device.

European Patent No. EP 2 378 305 B1 relates to a method and a system for localizing an object. The method of EP 2 378 305 B1 comprises emitting a magnetic field using a uniaxial source situated on one side of a magnetic device, resulting in a generation of a magnetic field by the magnetic device. The method further comprises measuring the magnetic field generated by the magnetic device. The emitted magnetic field is subtracted from the magnetic field generated by the magnetic device, and this enables determining a localization of the object.

A method for determining a position and an orientation of a medical instrument is disclosed in European Patent No. EP 2 034 879 B1. The medical instrument comprises a localization device with an antenna, and a circuit connected to the antenna. The method of EP 2 034 879 B1 comprises emitting electromagnetic radiation using a transmission unit, resulting in a generation of an electromagnetic radiation by the circuit of the localization device. A receiving unit senses the generated electromagnetic radiation, and this enables determination of the position and the orientation of the medical instrument.

US Patent Application No. US 2020/0397510 A1 describes a tracking system for tracking a marker device or localization device. The marker device comprises a casing within which is arranged a magnetic object. The magnetic object oscillates around its rotation axis when an external magnetic or electromagnetic excitation is acting on the magnetic object. A method for tracking a marker device is also described in US 2020/0397510 A1. The method comprises generating a magnetic or electromagnetic excitation field for inducing mechanical oscillations of the magnetic object, simultaneously transducing a magnetic or electromagnetic field generated by the induced mechanical oscillations of the magnetic object into an electrical response signal and determining a position of the marker device on the basis of the electrical response signals.

US Patent Application No. US 2022/0257138 A1 describes a tracking system for tracking a position of a marker device for a medical procedure on a patient's body. The marker device comprises a sensing unit that comprises a magnetic object providing a permanent magnetic moment. The magnetic object is attached to one end of an attachment portion, such as a filament, and the other end of the attachment portion is attached to the casing. The magnetic object is rotatable out of an equilibrium orientation by an external magnetic torque which is generated by an external magnetic or electromagnetic field acting on the magnetic object. The rotation of the magnetic object occurs around a virtual rotational axis centrally traversing the magnetic object which is rotationally symmetric with respect to the virtual rotational axis. The sensing unit further comprises a restoring torque unit. The restoring torque unit provides a restoring torque to force the magnetic object back to an equilibrium orientation if the external magnetic or electromagnetic field has rotated the magnetic object out of its equilibrium orientation. The tracking system comprises a plurality of coils which are configured to generate magnetic or electromagnetic excitation fields for inducing the mechanical oscillations of the magnetic object. The plurality of coils is further configured to transduce a magnetic or electromagnetic field generated by the induced mechanical oscillations of the magnetic object into a plurality of electrical response signals. The tracking system further comprises a plurality of transceivers configured to be connected to the plurality of coils. The tracking system further comprises a processor which is configured to determine the corresponding position of the marker device based on the one or more electrical response signals.

### SUMMARY OF THE INVENTION

This document discloses a small-scale magneto-oscillatory localization (SMOL) method and device which is capable of wirelessly locating, i.e., localizing a tracker, such as a millimeter-scale tracker. Using this method and device, the tracker can be localized in viscoelastic environments, such as biological materials, with all six degrees-of-freedom (6 DoF) over a large distance and without the use of radiation or RF signals. The device is a mechanically resonant structure which utilizes a single and finite magnetic moment in form of a magnet. The magnet is attached to a micro-cantilever and oscillates at a designed frequency around a rotational axis perpendicular to the single and finite magnetic moment to break the rotational symmetry of the magnet. The structure is excited by an excitation coil and a magnetic signal emitted after excitation can be sensed by an external sensor unit which can be evaluated for full six DoF localization with sub-millimeter accuracy and very high angular accuracy with a single sub-millimeter-sized magnet.

The structure can also be excited by providing a linear mechanical motion (in the form of longitudinal or transversal waves) in a direction substantially perpendicular to the long axis of the micro-cantilever and in the oscillation plane of the micro-cantilever. This motion excites the magnet on the micro-cantilever owing to the relative motion between housing and the magnet. The connection between the housing and the magnet is non-rigid, i.e., elastic, and has a finite length (corresponding to the cantilever length of the micro-cantilever) the relative motion inputs kinetic energy into the micro-cantilever which converts to elastic (potential) energy in the micro-cantilever by deflection of the micro-cantilever.

The micro-cantilever has a resonance frequency and, as would be expected, mechanical excitation of the micro-cantilever at this resonance frequency will lead to an increase of the oscillation amplitude with every motion of the housing. As the mechanical excitation does not necessitate a magnetic field for excitation of the resonant structure, this type of excitation does not interfere with the magnetic signal arising from the oscillation magnetic moment. Unlike in the prior art, there is no need for some kind of torsional motion to excite a purely torsional motion of the resonating structure the device.

The use of the magnet means that the device is compatible with common magnetic actuation schemes, allowing incremental robot tracking. The SMOL device combines a frequency encoding property of EM devices and the miniaturized footprint of the magnet. The alternating magnetic field generated by the oscillating micro-magnet is measured at multiple locations and fitted to a magnetic field model by a weighted Levenberg-Marquardt optimization algorithm, so that all three translational DoF and three rotational DoF of the device are accurately determined. The SMOL device can be readily integrated inside a helical millirobot, and the micro-magnet can be used for both localization and propulsion under different magnetic fields' excitations. The millirobot is tested in a biological gel phantom mimicking human brain tissues and a real porcine brain. The results show that full six DoF tracking of the millirobot is achieved with very high spatial resolutions, which are under sub-millimeter for three translations' DoF, sub-degree for the two rotational axes perpendicular to the cantilever and approximately 4° for the cantilever axis. The SMOL method requires simple instrumentation and exploits a unique frequency response of the device to maintain high signal-to-noise ratio (SNR) ratios in magnetically noisy environment. Localizing small-scale robots in deep human body for real clinical applications can be envisaged as a field application.

This document describes a method for determining a position and an orientation of a localization device, such as a tracker. The localization device has a magnet attached to an oscillating element located in the tracker. The magnet is excited using an external force or torque, such as an exciting magnetic field or mechanical wave and this results in a complex oscillatory motion of the magnet, which generates its own varying magnetic field. The complex motion comprises translation and rotation of the magnetic moment of the said magnet. The said complex oscillatory motion is a rotation of the magnet about a rotational axis, and the rotational axis is located an offset distance relative to the center of the magnetic moment of the magnet. The inventors found out that the offset distance enables an excitation of the magnet using mechanical waves, as highlighted above. The magnetic field generated by the magnet is sensed using at least one sensor and determining the position and the orientation of the localization device can be calculated from the sensing.

The mechanical wave can be oscillating in the longitudinal direction and/or in the shear direction of the oscillating element.

The rotational axis and the magnetic moment vector stemming from the magnet are non-parallel, and in one aspect substantially perpendicular to each other.

The oscillating element is located within a rigid or semi-rigid housing to protect the oscillating element from surrounding biological tissue.

In order to avoid saturation of the sensor, the sensing is performed when the excitation magnetic field is stopped.

The oscillating element comprises a restoring force unit of at least one of a cantilever beam or a similar unit which provides an elastic restoring force.

The exciting, the sensing and the determining of the position and the orientation of the device can be (continuously) repeated or be non-continuous.

The frequency of excitation of the magnet is approximately the resonance frequency of the oscillating element.

The method can be used to determine local viscoelastic properties of materials.

The sensing of the magnetic field generated by the magnet can be followed by determining local viscoelastic properties of material. The determined local viscoelastic properties of the material can be further used to determine the position and orientation of the localization device.

An apparatus for determining a position and an orientation of a localization device is also taught in this document. The localization device has a housing within which a magnet attached to an oscillating element is located. The apparatus comprises an excitation unit for exciting the magnet using one of an external force or torque resulting in a complex oscillatory motion of the magnet. The said complex motion comprises translation and rotation of the magnetic moment of the said magnet about a rotational axis located at an offset distance relative to the center of the magnetic moment of the magnet. The apparatus further comprises a data acquisition unit for sensing a magnetic field B, which is generated by the oscillating magnet, by using a sensor.

The magnet comprises a permanent magnet made of a magnetic material, preferably a ferromagnetic material.

The oscillating element comprises a restoring force unit, and in one aspect a cantilever.

The apparatus is used to localize a medical implant in an animal or a human body, chosen from one or more of a catheter, a stent, a guidewire, an endoscope, a capsule endoscope, a drug delivery device, or a small-scale robot, or to localize an anatomy chosen from one or more of a tumor, blood vessel, blood clot, polyp, nerve.

### DESCRIPTION OF THE FIGURES

FIG. 1A show an overview of the system with magnetic excitation.

FIG. 1B shows an overview of the system with mechanical excitation.

FIG. 2 shows a device with the cantilever at resting position and one deflected position.

FIG. 3A shows a schematic time sequence of F, θ and B.

FIG. 3B shows real data time signal timeline of F, θ and B.

FIGS. 4A-4D show a schematic diagram summarizing the four methods for determining the position and the orientation of the device.

FIGS. 5A-5C show results of the sensing of the device inside a housing.

FIG. 6 shows the device incorporated into an apparatus which comprises interconnected parts.

FIG. 7 shows a data evaluation flow chart for determining the position and the orientation of the device.

FIG. 8 shows a surface mesh of measured signal amplitudes in x direction for a tracker.

FIG. 9A shows a localization accuracy along the x- and z-axis for a translation of 50 mm and 25 mm along the respective axis of the tracker.

FIG. 9B shows an angular accuracy for rotations of the tracker around the intrinsic z-axis and γ-axis. FIGS. 9A-9B show experimental and simulations results.

FIG. 10 shows a simulation of an absolute depth error for an increasing depth with varying noise and magnet conditions.

FIG. 11 shows a signal-to-noise ratio for a B-field signal measured in x-directions for varying distances from sensors.

FIG. 12 shows the maximum localization depth over the resonance frequency of the device.

FIG. 13 shows a view of the housing of the device.

FIG. 14 shows the integration of the tracker into a milli-robot as a potential embodiment.

FIG. 15 shows the positional and angular errors of the device integrated into a milli-robot.

FIG. 16 shows an embodiment of the tracker inside the milli-robot being inserted into the gray matter in the cerebrum of a porcine brain.

FIG. 17 shows images of the tracker inside the milli-robot being inserted in brain tissue localized using ultrasound.

FIG. 18 shows the results of the amplitude difference of the magnetic field for a rotation of the tracker around the z-axis.

FIG. 19 shows a Maxwell diagram of the tracker in a viscoelastic medium.

FIG. 20 shows the damping coefficients of the magnetic signal of the device inside gelatin.

FIG. 21 shows the damping coefficients of the magnetic signal of the device inside ex vivo animal tissue.

FIG. 22 shows an example of a shape of a cavity of the device.

FIG. 23 shows a flow chart for determining the position and the orientation of the device.

### DETAILED DESCRIPTION OF THE INVENTION

The invention will now be described on the basis of the drawings. It will be understood that the embodiments and aspects of the invention described herein are only examples and do not limit the protective scope of the claims in any way. The invention is defined by the claims and their equivalents. It will be understood that features of one aspect or embodiment of the invention can be combined with a feature of a different aspect or aspects and/or embodiments of the invention.

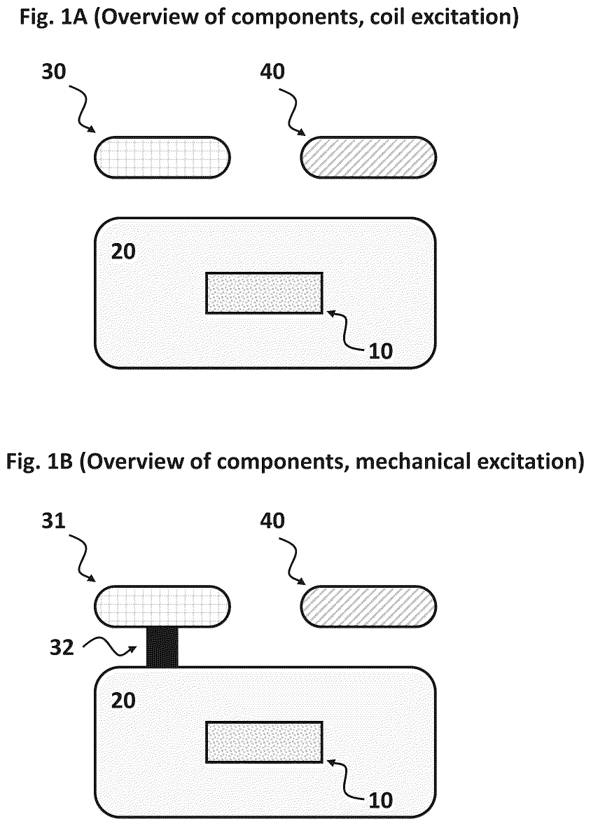

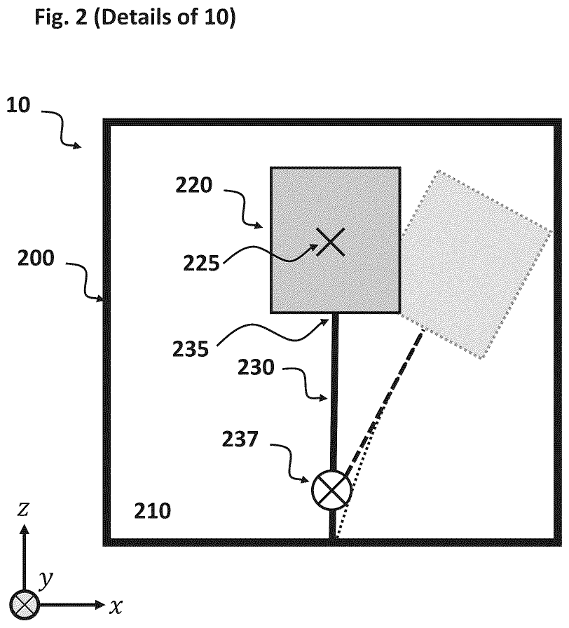

FIG. 1A shows an overview of the system for detecting a localization device or tracker 10 in (soft) biological tissue 20 using an excitation coil 30, supplied with a current, for generating an excitation magnetic field and a sensor unit 40 for measuring orthogonal components of a time-varying magnetic field Bx, By and Bz from the tracker 10. FIG. 1B shows an overview of the system for detecting a localization device or tracker 10 in (soft) biological tissue 20 using a mechanical excitation source 31 which is in physical contact over a transmitting part 32 for generating mechanical waves and a sensor unit 40 for measuring orthogonal components of a time-varying magnetic field Bx, By and Bz from the tracker 10. As noted above, the SMOL method is based on principles of mechanical resonance of a cantilever structure with an attached magnet 220 producing finite magnetic moment m, as shown in FIG. 2. The tracker 10 comprises a housing 200 with a cavity 210. In one non-limiting example, the cavity 210 has a rectangular shape. The cantilever structure comprises a cantilever 230 (or beam) with the magnet 220 arranged at a first end 235 of the cantilever 230.

The magnet 220 is formed of a permanent magnet, such as a ferromagnet, and acts as a transmitter of mechanical force from the excitation force F to the cantilever 230 and as an emitter of the varying magnetic field for sensing. In the following explanation, the magnetic flux density B of the varying magnetic field will be referred to as B-field or magnetic field and orientation descriptions will be given as extrinsic Euler rotation sequence z-x-y from the original orientation (0°,0°,0°) shown in FIG. 2 for the cantilever 230 in resting position (solid outline).

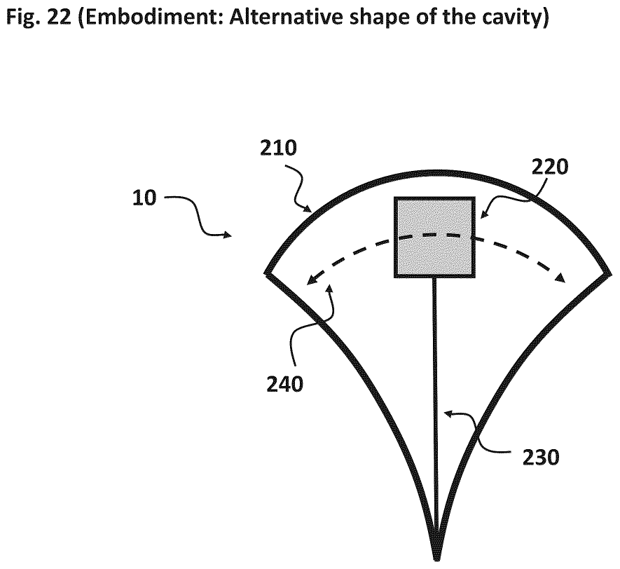

FIG. 22 illustrates a further example of the shape of the cavity 210. The inventors found out that optimizing shapes of the cavity 210 enable larger deflection angles θ of the magnet 220. The larger deflection angles θ result in a stronger signal of the magnetic field B, thereby resulting in a more accurate localization of the device 10. In one non-limiting example, it was established by simulation that localization depth, defined for a localization error <1 mm, increases by 20% by increasing the deflection angle from 10° to 20°. As the localization depth directly depends on the localization error, a higher accuracy at the same distance is obtained by increasing the deflection angle. Beyond 30° deflection angle, the percentual increase of localization depth, by increasing of the deflection angle, decreases greatly.

The shape of the cavity 210 as illustrated in FIG. 22 enables the larger deflection angle θ of the magnet 220 in comparison to the rectangular shape of the cavity 210 as illustrated in FIG. 2 for an identical inner volume of the cavity 210. The optimized shapes of the cavity 210 depend on the size and on the deformation of the cantilever 230, the size and the shape of the magnet 220, a movement path 240 of the magnet 220 and the field of application. Certain shapes of the cavity 210 are beneficial over others.

It was established that an “optimized” shape of the cavity 210 should be wider so the magnet 220 has more freedom to rotate and to translate within the cavity. There is, however, a trade-off between the size of the housing 200 and strength of the signal. This trade-off has to be established and depends on demands of the application. Tracking of a tool outside of the human body, e.g., scalpel, does not require a substantially small tracker 10. A larger tracker 10 with an optimized shape of the cavity 210 can be used. A smaller tracker 210 is advantageous for an intracorporeal, minimally-invasive application for the tracking of the tool from outside of the body. However, the smaller tracker has a cost of lower signal strength. The shapes of the cavity 210 are designed based on the movement path 240 of the magnet 220 on the cantilever 230, for example by optical recording. The movement path 240 is estimated by physical models, for example by beam theory, or by physical assumptions.

The magnetic field stemming from a magnetic moment m of 220 is approximated by the ideal dipole model:

$$B = \frac{{μ}_{0}}{4 π {‖ r ‖}^{3}} ( 3 \hat{r} \hat{r} - I ) m \qquad ( 1 )$$

which describes the components of the varying magnetic field B= [symbol] Bx, By, Bz [symbol] at a position r with respect to a dipole center 225 and m as the magnetic moment vector. \hat{r} is the normalized vector r, I am the identity matrix and μ0 is the permeability of free space. In relation to a point in space, in this case the sensor 40, the distance to the dipole center 225 of an arbitrarily oriented cantilever 230 can be described by

\vec{r}=\vec{r}₁+\vec{r}₂, (2)
where \vec{r}₁ is the vector between sensor 40 and rotation center 237 of the cantilever 230 and \vec{r}₂ is the vector between the rotation center 237 and the dipole center 225. \vec{r}₁ is constant for a fixed position of the cantilever 230, whereas the vector \vec{r}₂ is dependent on the current position of the magnet 220 at a time t. While the magnet 220 moves with the cantilever 230, a circular path with radius l₀ of the magnet 220 can be assumed and 2D polar coordinates in the xz-plane lead to

\vec{r}₂=l₀R_{q} [symbol] sin(θ(t),0,cos(θ(t)) [symbol] (3)
with l₀=|\vec{r}₂| being length between the dipole center 225 and the rotation center 237, and R_{q} being a 3D quaternion rotation matrix. The time-dependent Eq. 1 is completed with the magnetic moment at rest pointing in positive x-direction:

$$m ( t ) = {R}_{q} {R}_{y} \frac{{B}_{r} V}{{μ}_{0}} 〈 1 , 0 , 0 〉 \qquad ( 4 )$$

Matrix R_{y} describes a rotation around the y-axis for an angle θ as in a right-handed coordinate system, Bᵣ is a remanence field of the magnet and Vis the magnetic volume. A time-dependent oscillating dipole field therefore breaks the rotational symmetry of a static dipole around its magnetic moment axis by rotation around a perpendicular axis (as expressed by Eqns. 3 and 4), leading to a unique solution of Eq. 1 for the respective position r and rotation matrix R_{q}. Hence, all six degrees-of-freedom of the tracker 10 can be determined.

Under assumption of an external magnetic field Bₑₓₜ perpendicular to the magnetic moment m of the magnet 220, a torque r is applied on the magnet 220 which forces the magnetic moment m of the magnet 220 to align with Bₑₓₜ:

τ=m×Bₑₓₜ (5)

A torque τ is transmitted to the cantilever 230 which leads to a restoring torque (bending moment) and an angular deflection θ. Due to physical boundaries of the available oscillation volume within the cavity 210, the cantilever 230 with the magnet 220 is constrained to a maximum angle of θₘₐₓ.

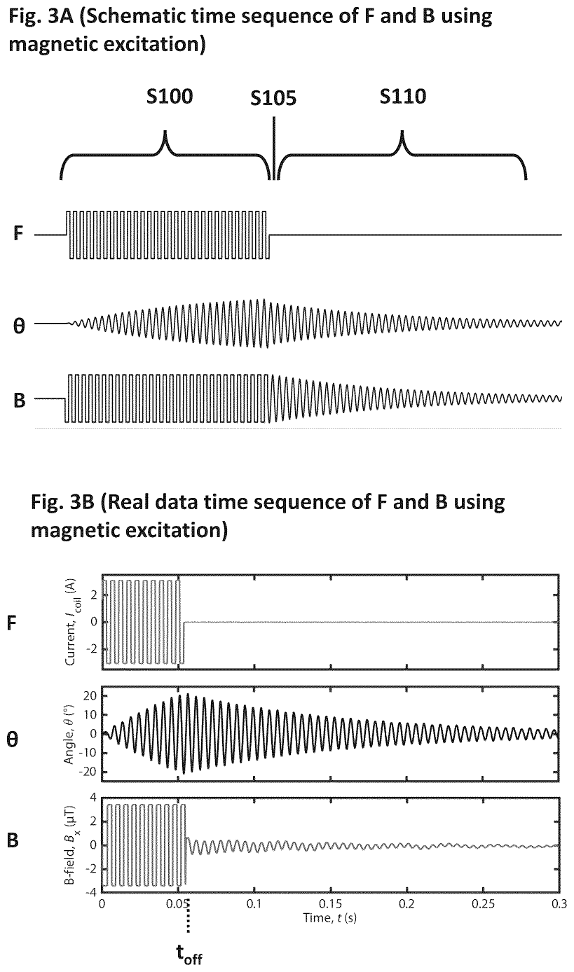

A schematic time sequence of a measurement at a single point for a single sensor 40 and magnetic excitation is shown in FIG. 3A. The measurement can be divided into an excitation phase (S100) and an evaluation phase (S110). In a first phase, coil current I_{coil} is passed through the excitation coil 30 which generates an excitation magnetic field and provides the system's energy input which drives the cantilever 230 at its resonance frequency, gradually increasing the deflection angle θ. Since the magnitude of the excitation magnetic field F is beyond the measurement range of the sensors 40, the sensors 40 saturate periodically. During the second phase, the excitation magnetic field F is shut off (S105) at time t_{off} and the stored energy in the cantilever 230 is released in an underdamped oscillatory movement, shown in the second line of FIG. 3A. The position of the magnet 220 moves in space, as shown in FIG. 2 (dashed outline) and explained by Eqns. 2 to 4. The movement of the magnet 220 results in emission of the varying magnetic field according to Eqn. 1.

The oscillation of the tracker 10 will now be explained using an example in FIG. 3B. It will be appreciated that the values are not limiting of the invention and serve only to illustrate the invention. The cantilever 230, shown in FIG. 2, was magnetically excited at its resonance frequency of 187 Hz by 10 square wave pulses in the excitation coil 30 and recorded with a high-speed camera. The magnetic signal Bₓ (i.e., x-component of the varying magnetic field from the magnet 220 oscillating on the beam) at one of the sensors 40 in x-direction and the current in the excitation coil I_{coil} were measured for 0.3 s. In FIG. 3B, the value of I_{coil}, the angular deflection θ and the one corresponding value of the magnetic signal Bₓ are compared. In the beginning of the measurement, the external magnetic excitation field F leads to a saturation of the magnetometer in the sensor 40 for which the saturation field is reached at ±3.5 μT. A direct increase of θ can be seen which was evaluated visually at 5000 frames-per-second by image analysis. After the excitation stops at time t_{off}, an exponential damping of θ can be observed which can be mathematically described as damped harmonic oscillator with frequency f and damping coefficient η:

θ(t)=θₘₐₓ sin(2πft)exp(−ηt) (6)

Similarly, the magnetic signal in direction i and position j can be described as:

Bi,j(t)=A sin(2πft+φ)exp(−ηt)+C (7)

with amplitude A, phase shift φ and offset C. The offset C is an invariable value which does not change and depends on a number of factors, including the geometry of the system. The approximation in Eqn. 7 is suitable when the lengths of the vectors \vec{r}₁>>\vec{r}₂₂, and the values of the B-field components do not change sign (as this leads to double-frequency distortions in the magnetic signal).

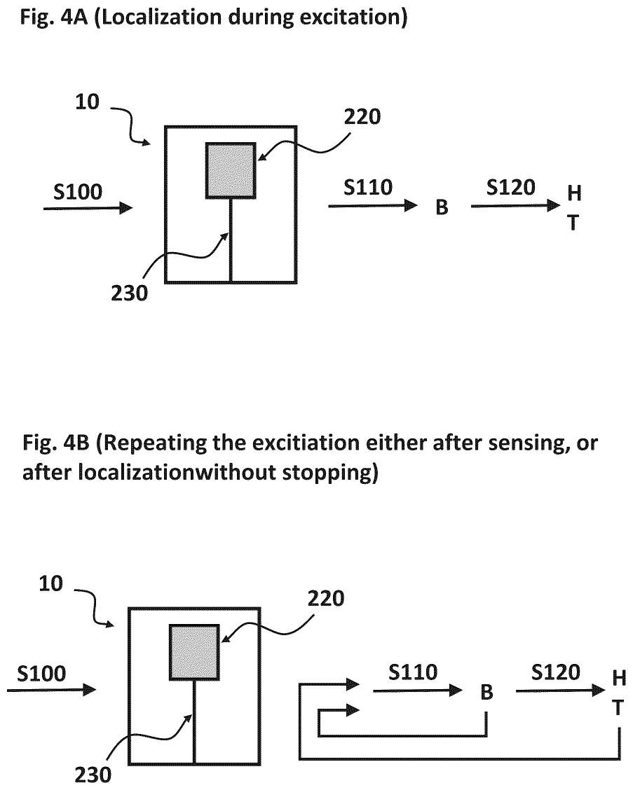

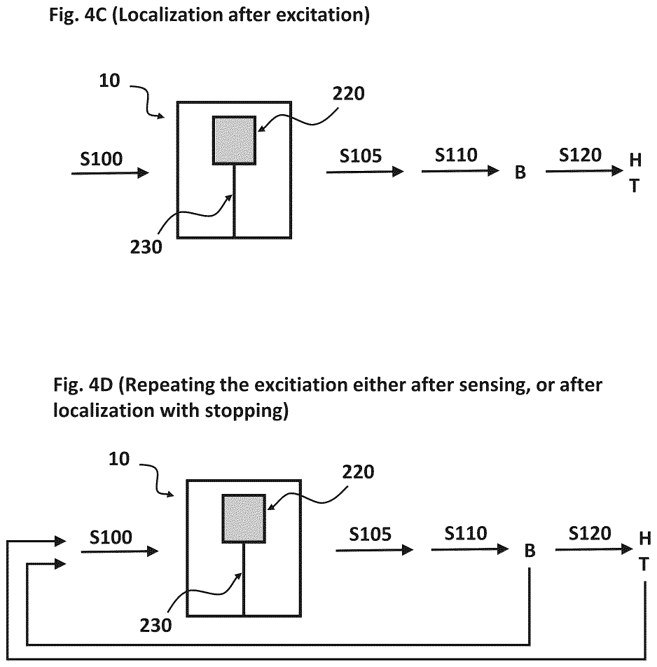

FIGS. 4A-4D show schematic diagrams summarizing the four methods for determining the position H and the orientation T of the tracker 10. All four methods comprise three main steps: exciting the magnet 220 in an exciting step S100 using an external force or torque F which results in a complex oscillatory motion of the magnet 220, sensing the varying magnetic field B generated by the magnet 220 in step 110 and deriving the position H and orientation T of the tracker in step 120.

The exciting of the magnet 220 in the exciting step S100 can be continuous, meaning that the magnet 220 is oscillating continuously, and at one moment in time, the magnetic field B is sensed (S110) and H and T are determined, as shown in FIG. 4A. As shown in FIG. 4B, the sensing in a sensing step S110 of B can be repeated during the continuous excitation in the exciting step S100, enabling to determine in determination step S120 repeatedly H and T. A sensing in step S110 of B can also be repeated after H and T are determined in the determination step S120.

The exciting step S100 can be non-continuous, meaning the excitation is stopped in step S105 and the complex oscillation of magnet 220 decays over time. The magnetic field B is sensed in the sensing step S110, and H and T are determined, as shown in FIG. 4C. Since the excitation was stopped in the step S105, the exciting step S100 is repeated after the sensing step S110 or after the determination step S120 for determining the position H and orientation T of the tracker 10, shown in FIG. 4D.

Repeating the sensing step S110 and the determination step S120 of the position H and orientation T of the tracker 10 after continuous (FIG. 4B) or non-continuous (FIG. 4D) exciting in the exciting step S100 enables the determination of the position H and the orientation T of the tracker 10 over time. The non-continuous excitation step S100 and the following steps S105, S110 and S120 can be carried out in e.g., periodic timesteps or when desired.

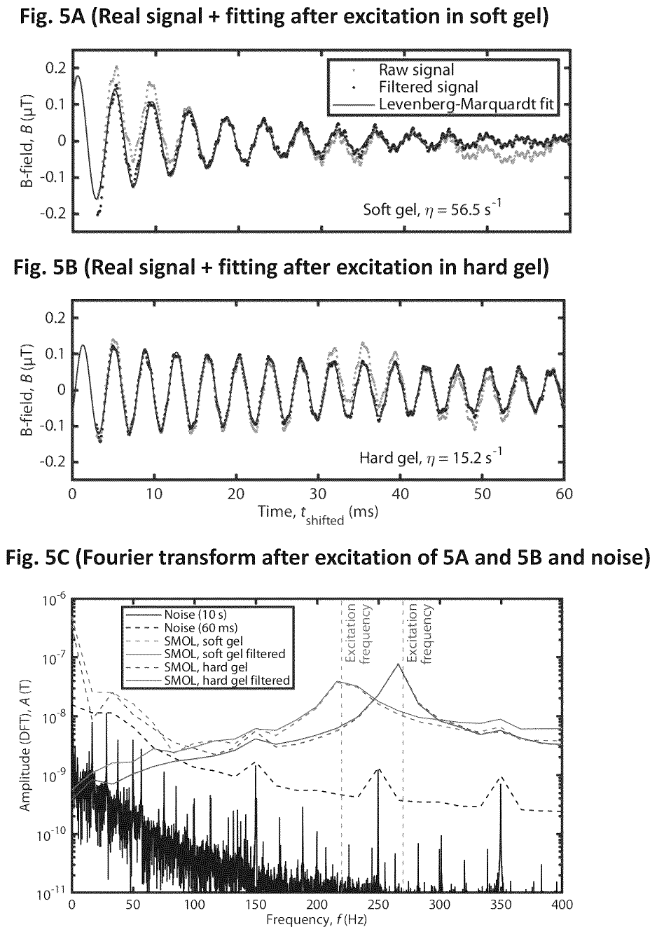

FIGS. 5A to 5C show results of the sensing step S110 for an exemplary tracker 10 inside a housing 200. The magnetic signals B were recorded in a soft and hard gelatin-agarose hydrogel over 60 ms. FIG. 5A shows the measured Bx component of the varying magnetic field B after the excitation (t_{shifted}=0) in FIGS. 5A and 5B in the time domain and in FIG. 5C in the frequency domain.

A small buffer of 3 ms between the excitation end and the evaluation start is applied as excitation coil cool-down. It can be seen that the raw signals exhibit low frequency noise which can also be seen in a discrete Fourier transform (DFT) in FIG. 5C. A high-pass filter at the (higher) resonance frequency was applied, shown as the filtered signals.

To this signal, Eq. 7 can be fitted using a Levenberg-Marquardt algorithm to extract the features of the damped sine curve (FIGS. 5A and 5B, solid line). Due to the damped behavior, peaks in the DFT (FIG. 5C) appear broad and the frequency resolution with 17 Hz is very low, making an analysis in the frequency spectrum inaccurate. FIG. 5C additionally shows a 10 s recording of the noisy magnetic environment in which all tests were performed. The strongest noise amplitudes with up to 56 nT were measured at 16.7 Hz, 28 Hz and 50 Hz. Prominent frequencies above 100 Hz are about 2.5 nT, which induces limitations to the localization distance.

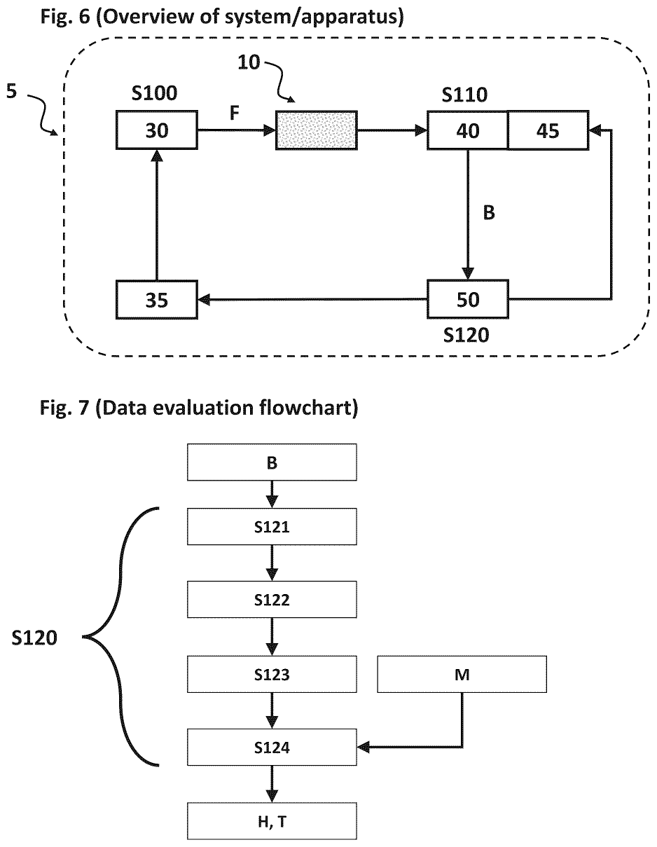

The tracker 10 can be incorporated into an apparatus 5 which comprises the interconnected parts as shown in FIG. 6. Here, for the purpose of demonstration, excitation using a magnetic excitation field is presented. Iterative device control and data processing was performed in MATLAB.

The sensing unit 40 included three fluxgate sensors that were arranged orthogonally with a custom printed element and attached to a 2D positioning stage 45 over a 75 cm long rod to reduce magnetic influence from the electric motors driving the positioning stage. The sensors 40 are moved to locations in a grid pattern in the xy-plane. At each position, a predefined excitation signal at the estimated resonance frequency fᵣₑₛ is sent to a current amplifier 35 and further to the excitation coil 30 with the current I_{coil}. The alternating current (AC) induced into the excitation coil 30 generates the AC magnetic field which is able to excite (S100) the mechanically resonant structure of the tracker 10 by the excitation force F, as described above.

An exemplary signal evaluation procedure S120 is shown in FIG. 7. It will be appreciated that the recorded sensor signal by the sensor 40 includes the excitation signal from the excitation coil 30 and thus, initially, the evaluable signal is separated at a cut-off time. The cut-off time is dependent on the behavior of the excitation coil 30 and the individual configurations of the sensor 40. A moving mean with a window length dependent of f_{exct} is calculated which acts as a low-pass filter S121. From the raw signal B, the moving mean is subtracted to obtain the high-pass signal which includes the resonance frequency signal of the cantilever 230.

A Levenberg-Marquardt algorithm is deployed to fit Eq. 7 to the signal to acquire the relevant free parameters (S122) of Eq. 7. Utilizing parameters such as the frequency, the damping coefficient, or the coefficient of determination, the in step S122 obtained signal amplitudes can be further filtered in step S123. Thresholds for physically unreasonable values in step S123 from step S122 can be, for example, defined fixed values.

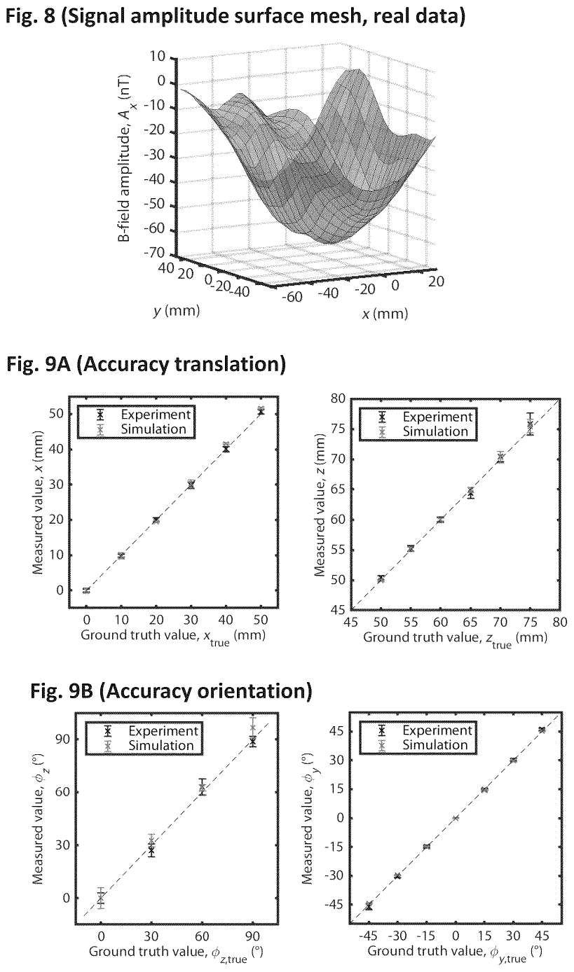

All through S121 to S123 acquired amplitudes A, exemplarily shown in a surface mesh FIG. 8 in the x-direction, are used as input for the final localization step S124. The mathematical model M described above with fixed cantilever length l₀ of the cantilever 230 and the constant magnetic moment m (t=0) of the magnet 220 is used in a weighted Levenberg-Marquardt algorithm with cost function:

$${J}_{i \cdot j} = {\sum }_{i = 1}^{3} {( {A}_{i}^{*} - {\hat{A}}_{i} )}^{T} diag ( {W}_{i} ) \cdot ( {A}_{i}^{*} - {\hat{A}}_{i} ) \qquad ( 8 )$$

and weighting matrix W, where Â is the optimization amplitude matrix of the magnetic signal calculated at sensor position j by

$${\hat{A}}_{j} = \frac{{B}_{j}^{- θ} ( p ) - {B}_{j}^{+ θ} ( p )}{2} \qquad ( 9 )$$

with p being the vector of optimization parameters including the position x, y, z, the orientation, given in quaternions, q₀,q₁,q₂,q₃ and the deflection angle θ. The parameters are randomized within physically reasonable ranges and the optimization algorithm is repeated a fixed number of times to avoid outliers. After automatic selection of the best fitting parameters p, the position H and orientation T of the tracker 10 are obtained.

In order to demonstrate the localization accuracy, both real experiments and simulations using model M have been conducted. Since a visual ground truth with respect to the measurement plane is difficult to establish with an accuracy below 1 mm, differential measurements were performed.

FIG. 9A shows the localization accuracy along the x-axis and z-axis for a translation of 50 mm and 25 mm along the respective axis. Along the x-axis, the localization values perfectly match the ground truth values over the whole 50 mm range, which is half of the total 100 mm×100 mm scanning plane, with an average accuracy of 0.6 mm±0.6 mm and the largest difference from the ground truth is 0.6 mm at the edge of the scanning area. Since the scanning area is symmetrical along the x- and y-axes, respectively, the errors in the directions of −x, −y, +y are expected to be similar as +x. On average for the z-axis, over the z distance between 50 mm and 75 mm, the accuracy is 0.7 mm±0.9 mm. Here, a decrease of the accuracy can be noticed at a larger distance, as the magnetic field decays over the cube of the distance according to Eq. 1 Below 65 mm distance, the accuracy is 0.5 mm±0.6 mm. Overall, the translational accuracies in all directions are significantly below 1 mm, revealing the high accuracy of the SMOL method.

FIG. 9B shows the angular accuracy for rotations around the intrinsic z-axis and y-axis (FIG. 2), respectively. In order to determine angular deviations, an axis perpendicular to the rotation axis is taken as reference and circular statistics are used to calculate the standard deviation. For rotations around the cantilever axis, i.e., the z-axis (FIG. 2), the accuracy amounts to 3.4°±3.7°, whereas for rotations around the y-axis, the accuracy is significantly better with 0.7°±0.8°. Due to orthogonality of the rotation axes, a similar accuracy can be expected for rotation around the magnetic moments axis (x-axis). Overall, the cantilever axis shows more variation in comparison with the other two axes perpendicular to the cantilever, but all rotational localization accuracies are significantly lower than 5°.

Simulation results are also shown in FIG. 9A and B and match very well the experimental results for all accuracy measurements. It reveals that the numerical model clearly represents important features of the SMOL method, as it considers magnetic noise in all directions. The agreement between simulation and experiments validates the numerical model, and thus the latter offers a valuable way to predict and optimize the performance of the trackers 10 with a decreased size and an increased measurement distance.

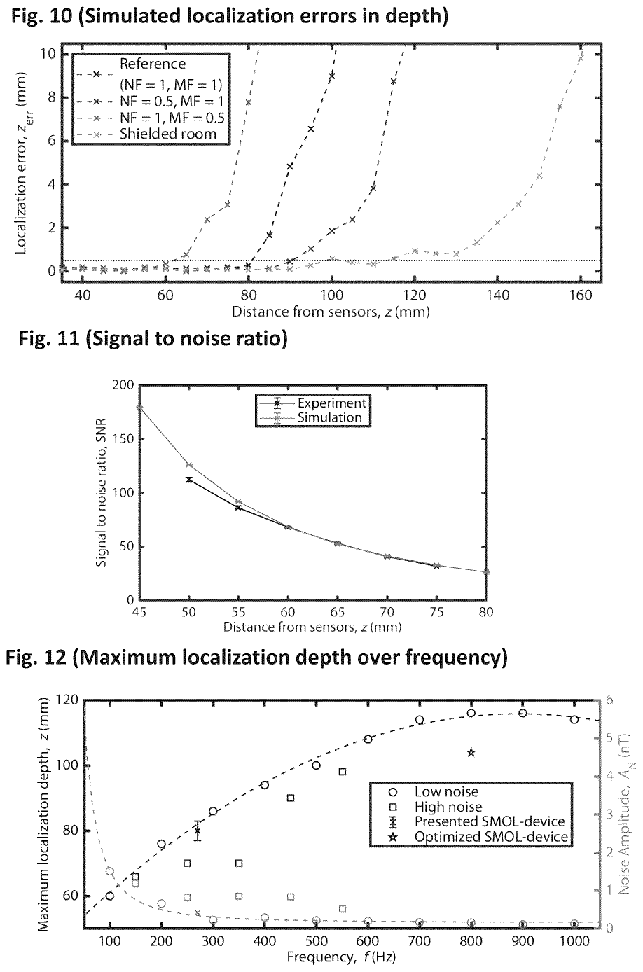

To gain insight on the performance of the SMOL method under different magnetic noise conditions, the absolute depth error zₑᵣᵣ is simulated for an increasing depth z with varying noise and magnet conditions. As shown in FIG. 10, the reference curve (black) represents the system with a noise factor (NF) and magnetic moment factor (MF) both of I, meaning that the noise and magnetic moment used for simulations are the same as those in the experimental settings. The impact of halving the noise (NF=0.5, MF=1) or the magnetic moment (NF=1, MF=0.5) is shown. It is observed in all curves that the localization errors stay at very low (sub-millimeter) level at close distances, but dramatically increase above a certain threshold. The maximal localization distance is defined as the distance when the localization error first reaches 0.5 mm (dashed horizontal line). The simulation results reveal that the tracker with half of the magnetic moment can be accurately localized up to 65 mm distance, and attenuating the magnetic noise to half can increase the distance to 90 mm. In a magnetically shielded room (only the electronic noise of the magnetic sensor is considered), a maximum of 110 mm detection distance can be achieved theoretically. Overall, operating in a magnetically shielded or low-noise environment and using even more sensitive magnetometers (40) can significantly increase the localization depth beyond 100 mm, making the SMOL method suitable for real clinical localization tasks, for example small-scale robots in deep human body.

The signal-to-noise ratio (SNR) is presented for the B-field signal measured in x-directions for varying distances from the sensors z in FIG. 11. Since a high-pass filter S121 is applied on the damped sinusoidal signal, the pure noise is treated similarly. Without removal of frequencies below 270 Hz, which is the tracker's resonance frequency, the standard deviation of the noise amounts to 20.9 nT, with filtering it drops to 1.7 nT. The latter value is used for the evaluation, and the amplitude A is the highest measured amplitude after filtering. As shown in FIG. 11, the SNR in simulation is above 170 at 45 mm distance and 25 at 80 mm distance, and the trend matches well to the model of the cubic decaying of the magnetic field over distance. The good fit to the experimental data again indicates the correct modelling of the physical process in the simulation.

Hence, the simulation reflects the real system with high accuracy, and in other words, the real system behaves sufficiently idealistic to be simulated numerically and further characterizations of the tracker 10 are carried out numerically.

The frequency scalability of the SMOL method is presented in FIG. 12. The maximal localization distance (black), using a cut-off threshold of 0.5 mm localization error (horizontal dashed line in FIG. 10), was simulated for frequencies ranging from 100 Hz to 1 kHz using recorded noise (FIG. 5C). A significant increase of the localization depth by increasing frequency can be observed. For example, for a Tracker 10 at 100 Hz, the maximal localization distance is around 60 mm, and for a device at 800 Hz, the distance almost doubles to 120 mm. The main reason is that the magnetic noise (from the environment and the sensing electronics) A_{N} (gray) has an overall dropping amplitude over the frequency. Noticeably, there are strong noise amplitudes (shown as the gray squares) at particular frequencies, such as 250 Hz, 350 Hz and 450 Hz. The localization distances (black squares) are severely impeded by the strong noise at these frequencies. Hence, the optimal operational frequency for the SMOL method should be chosen where the environmental and sensor-inherent noises are minimal.

The increase of the resonance frequency will not only result in enlargement of the maximal localization distance, but also enable the further downscaling of the Tracker 10. Using a two-dimensional geometric model of the spatially constrained cantilever 230 (FIG. 2), a miniaturized and optimized Tracker 10 with a total volume of 1 mm³ is designed and simulated. The result is shown as a black star in FIG. 12. It has a magnetic volume of 0.4 mm³ (1 mm×0.8 mm×0.5 mm), a maximum deflection angle of θ=17° and a cantilever 230 length of 0.5 mm. In order to obtain a resonance frequency of 800 Hz for such a device, a steel cantilever with approximately 5 μm thickness and 1 mm width is required. Applying MEMS fabrication techniques, it is feasible to construct such a device, which will enable sub-millimeter accuracy, full 6 DoF localization at a distance of 100 times of the tracker size. Therefore, the SMOL method can open up unprecedented possibilities for the localization and tracking of sub-millimeter scale implants, including small-scale robots, in deep human bodies.

The method set out in this document uses a magnet 220, which can also be used for the magnetic actuation of small-scale robots. During actuation, a magnetic torque (Eq. 5) around the cantilever axis is applied to the micro-magnet 220 and the cantilever 230 is able to transfer the torsion to the housing 200, which leads to rotation of the tracker 10.

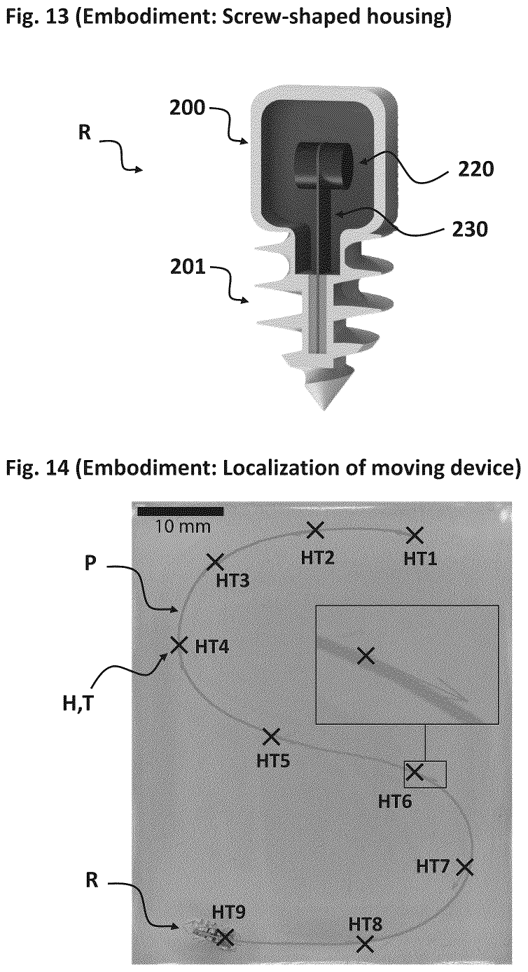

In one embodiment of the presented method and device, the housing 200 can have a helical or screw-like shape 201 on the surface, as shown in FIG. 13, to couple the rotation to translation, and thus propel in soft viscoelastic materials. Special care was taken in the robot R design to avoid damaging the thin cantilever 230. If the magnet 220 is free to rotate without angular restriction, the strong magnetic torque will keep twisting the cantilever 230 and exceed the strength limit of the cantilever material leading to a permanent plastic deformation and the fracture of the beam 230. Geometric constraints inside the housing 200 (see FIG. 13) are added to transmit the torque by direct contact to the housing 200.

In one embodiment, the tracker 10 is integrated into a milli-robot R moving on a path P as demonstrated in FIG. 14. Actuation of the screw-shaped robot R was performed with a rotating permanent with two rotation axes. The actuation axis, in the case of forward propulsion, has to be aligned with the cantilever axis. In order to turn the robot R, the actuation magnet needs to be rotated around the steering axis. Since simultaneous actuation and localization is currently not possible with the SMOL method, the measurement was performed incrementally (HT1 to HT9), meaning that the container was transferred between the actuation setup and the localization setup.

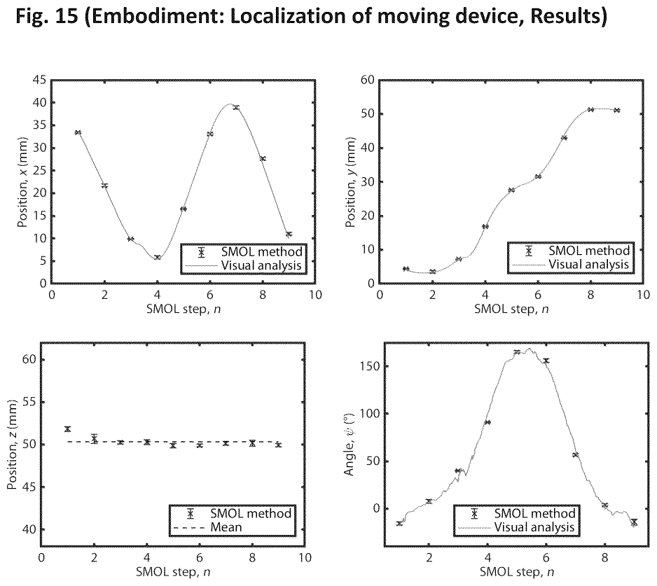

A detailed analysis shows that the in-plane positional errors (x, y in FIG. 15), obtained at 50 mm distance from the sensors 40, are on average 0.2 mm±0.1 mm comparing to the visual ground truths. The z-position is stable around an average value of 50.3 mm±0.6 mm. Angular measurements of the in-plane angle ψ amount to an average error of 4.7°±3.6° with a standard deviation of 1.5° on average for all measured points. These results show the accurate localization possibility with the presented method; moreover, they also demonstrate the possibility of using the same magnetic moment m on the miniaturized robot R for both actuation and localization purposes. In one embodiment, the tracker 10 or robot R can be inserted into biological tissues, for example brain, which is known as one of the softest tissues in the human body. Strong mechanical damping behavior is expected to impede the mechanical oscillators in brain tissues, which creates an additional challenge to obtain a sustained oscillating signal of the tracker 10 for an accurate localization.

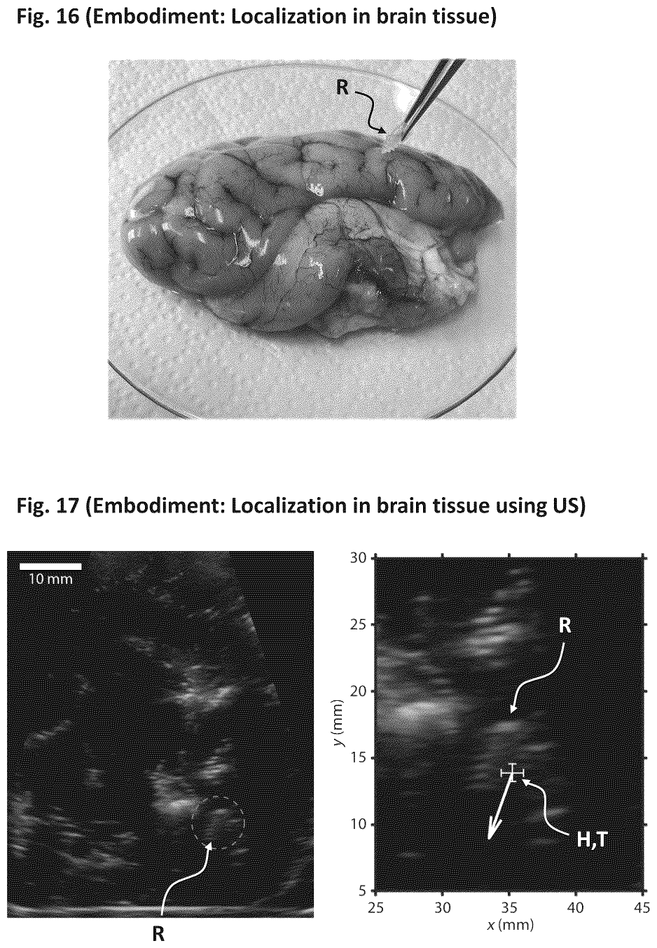

Therefore, brain tissues were chosen as a realistic and strict testing environment for the presented method. The results of the presented method working in an ex-vivo porcine brain are shown in FIG. 16. It shows half of a porcine brain and the tracker 10 inside robot R being inserted into the gray matter in the cerebrum. US imaging is used to obtain planar information of the location H of the tracker 10 relative to the rigid boundaries of the container.

As shown in FIG. 17, the overall US imaging resolution and contrast are poor due to the multiple reflection and scattering of the US beam in inhomogeneous brain tissues. The robot R (circled) at a distance of 40 mm to the US probe is barely distinguishable from the background noise. On the contrary to US imaging, the SMOL method accurately detects the position H and orientation T of the robot R in the brain. As shown in FIG. 6C, the localization information H, T is overlaid with the US image, and very good correlation is found between the two localization methods. At 45 mm z-distance from the magnetic sensors 40, the standard deviation of 10 independent measurements is 0.9 mm in x-direction and 0.7 mm in y-direction. It reveals the high reproducibility of the presented method in biological soft tissues. The white arrow indicates the detected orientation H of the robot's R major axis, which aligns very well with the estimated orientation by US imaging. A shift of approximately 3 mm in y-direction is seen. This systematic error may be caused by the deformation of the soft tissue under the compression of the US probe, as the probe must be in close contact through ultrasound gel with the brain tissue during imaging. It indicates another important advantage of the presented method over US imaging that it is a wireless localization method that does not require any mechanical contact with the biological tissues, which can be a crucial aspect to protect the delicate soft organ, such as the brain, in real clinical applications.

The presented method offers a completely wireless localization technique that requires no physical contact of the external devices 30 and 40 to the soft tissues 20, which will benefit minimally invasive and robotic surgeries, where a direct contact of the imaging probe to the internal organs is often not possible. The minimal incision required to insert a tracker 10 into the body is very small, which can be readily introduced via a needle, a catheter, or an endoscope. It exhibits a small footprint and requires no onboard power, which makes it easier to be integrated with wireless medical devices, such as capsule endoscopes and implants. The high SNR (FIG. 11) and high accuracy over a large distance (FIG. 10) as well as its small size (FIG. 12) are beyond the possibilities of other wireless tracking methods, such as the methods based on static permanent magnets. The tunable, unique frequency response of the tracker 10 facilitates the isolation from DC and low frequency magnetic noise, i.e., surgical tools that are magnetic.

In one embodiment, unique frequency response of the tracker 10 enables the identification of multiple trackers 10 in the frequency space for multi-target 6 DoF simultaneous localization.

The presented method breaks the rotational symmetry of a static magnetic dipole by oscillating the magnetic moment around an axis perpendicular to the magnetic moment axis. Pure rotation of the magnetic moment, i.e., l₀=0, means that the difference of the B-fields from the two peak deflections −θ and +θ according to Eqs. 1 to 4, creates a new dipole field which can be measured at the resonance frequency of the tracker 10. This mathematical consideration implies that only 5 DoF can be determined, identical to the 5 DoF of a static magnetic dipole.

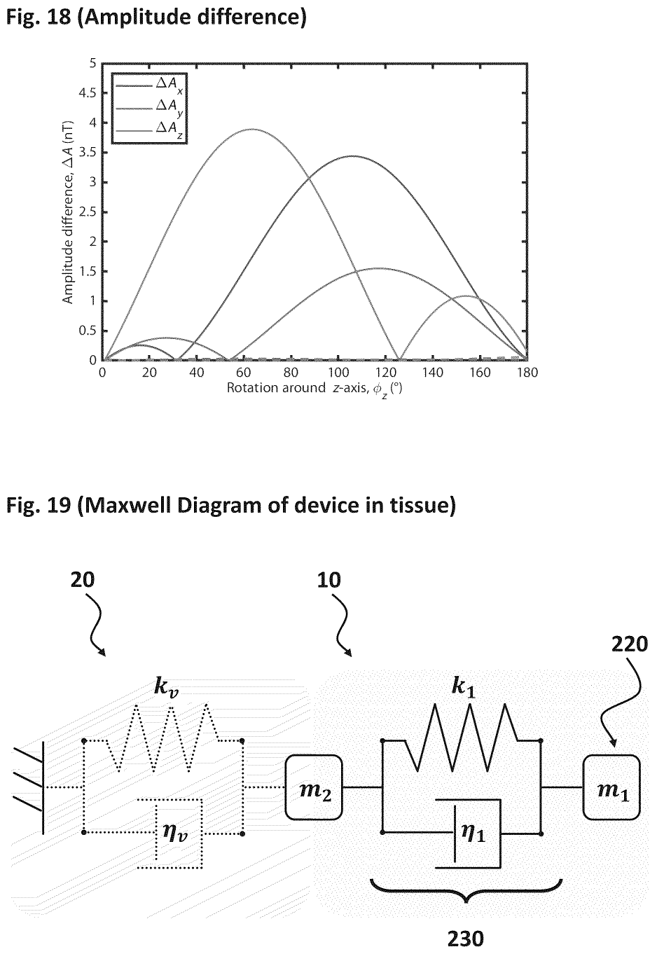

To overcome this limitation, translation is added to the rotation of the dipole moment, l₀>0 (FIG. 2), and the new dipole field cannot be described as a 5 DoF dipole anymore, hence, the symmetry around the rotational axis of the dipole broken. The difference between these two cases is shown in FIG. 18, for the case with cantilever 230 in solid lines and without cantilever 230 in dashed lines. The tracker 10 is rotated around the z-axis and only if there is a cantilever 230 to offset the oscillating magnetic moment m (FIG. 2), an amplitude difference of the oscillating magnetic field at nanotesla range can be detected, while without cantilever 230, the amplitude difference is close or equal to zero. The high asymmetry of the amplitudes for all rotations between 0° and 180° reveals the amplitude-encoding feature for 6 DoF determination using the presented method.

Only the amplitude difference, but not the absolute DC magnetic field, is considered as useful information in the measurement, since the DC field is subject to high noise and not robust enough to be detected for such a small device at a large distance.

Another way of establishing the 6 DoFs is to capture the double-frequency components of the oscillating B-field. Due to the high spatial non-linearity of dynamic B-field components of magnetic dipoles, the amplitude field of the oscillating dipole has zero-crossings. If a sensor position lies in proximity to such a zero-crossing, the B-field signal shows components at twice the resonance frequency. However, these features can only be reliably measured when the B-field is tightly scanned. In comparison, the presented method comprising a cantilever 230 offers a unique and more convenient way to detect all 6 DoF with much fewer magnetic sensors 40 and without reliance on such features.

Owing to the use of a single excitation coil 30, not all orientations of the tracker 10 can be sufficiently excited to achieve large deflection amplitudes. These blind spots occur at orientations where the magnetic torque t (Eq. 5), applied on the cantilever 230 through the external excitation field F (FIG. 3), is close or equal to zero. This is the case when the external field is parallel to the magnetic moment axis. In-between perpendicular and parallel and alignment, t decreases gradually, such that the maximum deflection is not always achieved. In order to cover all orientations, a total of three excitation coils 30 in orthogonal arrangement are necessary and the direction of the superpositioned B-field should preferably be parallel to the previously detected orientation T of the cantilever 230. Omnimagnets could be suitable for this application.

The presented system (FIG. 6) is a 2D scanning system that covers 25 points within a square with 10 cm side length in the xy-plane. Due to the fluxgate sensor 40 range of approximately ±3.5 μT, the sensors are easily saturated when a strong ferromagnetic object is in proximity to the scanning area. The manual B-field compensation function embedded in the sensor 40 is only used once in the center of the scanning area, to compensate the external B-fields. If the B-field gradient along the scanning plane is larger than the stated range, the signal cannot be picked up with this process. In the future, automatic DC magnetic field compensation at each position can be performed, or a static sensor array 40 can be used.

Since the tracker 10 is a mechanical system, it can change its properties over time and usage. The material used for the cantilever 230 (C1095) with very low chromium percentage is especially prone to corrosion. In can be expected that oxidation weakens the cantilever 230, since decreases of the resonance frequencies over time have been observed in many prototypes. However, all cantilevers 230 are encapsulated airtight and rarely used devices showed much less or no reduction of the resonance frequency over time. Other possible explanations could be the loosening of the fixed end of the cantilever from glue deterioration resulting in a longer beam l₀ or a reduction of the Youngs modulus E of the cantilever from dynamic fatigue.

The determination in the determination step S120 of the position H and the orientation T of the device 10 is performed, in one example, by a Fourier analysis in a step S200 of the signal of the magnetic field B to obtain the amplitudes A of the magnetic field B in the frequency domain in a step S210. The position H and the orientation T are determined from the magnitude of the amplitudes A of the magnetic field B between the sensor 40.

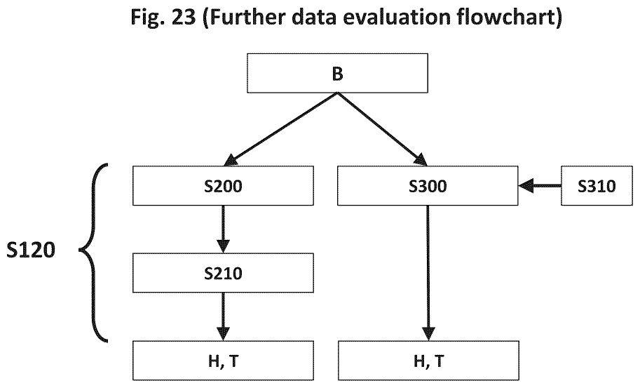

The determination in the determination step S120 of the position H and the orientation T of the device 10 is performed, in another example, by using in step S300 a physical model of the oscillations of the magnet 220. FIG. 23 shows that the determination step S120 comprises directly evaluating in step S300 the signal of the magnetic field B in the time domain. The physical model uses in a step S310 known values or calibrated values of the magnetic moment m of the magnet 220, the offset distance between the center of the magnet 220 and the rotation center, which are required for the step S300. Unlike in the prior art, the magnet 220 does not rotate around its own axis so the dipole center 225 is not the rotation center of the magnet 220. The center of the magnet 220 is in most cases its center of mass. The physical model further uses in the step S300 a maximum deflection angle θ, a signal damping ratio, the number and the position j of the sensor 40. Further physical parameters, for example, air resistance, the moment of inertia, the elastic modulus of the beam, etc., can be added to refine the physical model.

The use of the physical model over the Fourier analysis enables a reduction of a necessary recording time of signal required for localizing the device 10. This will now be explained. Determining spectral components of the signal in the frequency domain require a large number of periods, e. g, 10 or 40, to obtain a peak sharpness of the signal sufficient for accurately localizing the localization device 10. The physical model enables reducing the necessary recording time of signal to an integer number of a half-periods N of the oscillation, and possibly also to a fractional number of a half-periods N of the oscillation. The oscillatory motion and the resulting recorded signal are well-defined for any position T and any orientation T by using the physical model.

The physical model is fitted in step S300 to a time domain signal which results in optimized parameters of position H and orientation T. The time domain signal comprises the half-periods N. An algorithm, for example the least-squares method, minimizes in step S300 the error between the physical model of the signal and the recorded signal B. The minimum error is obtained for the optimized parameters, thereby obtaining the accurate position T and the orientation H of the localization device 10. The localization accuracy of the localization device 10 is further improved by increasing the number of half-periods N due to averaging of random noise from the environment.

The time domain signal is, in a further example, segmented into a desired integer number of half-periods N_seg. The desired integer number of half-periods is dependent on the degree of accuracy that is required. Only a single excitation is therefore sufficient for localizing the localization device 10 at multiple times.

For example, the localization device 10 with a resonance frequency of f=100 Hz can be localized at a rate of a double resonance frequency, i.e., 200 Hz, when every half-period N is evaluated independently (N_seg=1). The localization rate can be 100 Hz when two half-periods N are evaluated per segment, i.e., N_seg=2. The localization rate can be 50 Hz when four half-periods N are evaluated per segment, i.e., N_seg=4. An increase in the number of the half-periods N_seg results in a decrease of the maximally achievable localization rate to 2*f/N_seg. Very high velocities of the localization device 10, for example >200 mm/s, or very precise movement paths P are measured with the segmenting method. The signal decays over time, thereby the signal strength and the localization accuracy decrease until the localization device 10 needs to be excited again. A continuous or weakly damped oscillation of the magnet 220 results in a continuous or weakly damped signal and is therefore preferred over non-continuous or highly damped signals to avoid pauses for re-excitation in which the localization device 10 cannot be localized.

The localization device 10, i.e., the tracker, can be localized during excitation if the magnetic field from the excitation coils does not saturate the sensor 40. The localization device 10, i.e., the tracker, can further be localized during excitation if the magnetic field stemming from the excitation coils at the sensor 40 is zero or very low.

A further method for localizing the localization device 10 is mechanically exciting the localization device 10. The mechanical excitation does not interfere with the sensor 40 and allows continuous localization, i.e., tracking, due to a continuous excitation of the localization device 10 without pauses.

### EXAMPLES

Localization system devices. Iterative system control and data processing was performed in MATLAB (R2020b, The Math Works, US). For signal emission and analog signal conversion, a data acquisition board (USB-6343, NI, US) with an input range of ±11 V and 16 bit resolution, which corresponds to 0.33 mV resolution, was used. The weak magnetic fields were measured with three fluxgate sensors (Fluxmaster, Stefan Mayer Instruments, Germany) arranged orthogonally with a customized 3D-printed holder. They are provided with a manual offset compensation function and a sensitivity level of 1 V/μT, a range of approximately ±3.5 μT, a resolution of 0.1 nT and an inherent noise of 20 pT Hz^{-1/2} at 1 Hz. The sensor holder is attached to a 2D robotic positioning stage (M-414.2PD, 0.1 μm step size, PI, Germany) over a 75 cm long non-magnetic rod (Polymethyl methacrylate) to reduce magnetic influence from the electric motors. Using this positioning stage, 25 points in a 5×5 point grid pattern in the xy-plane were scanned. For the excitation of the tracker 10, a customized electromagnetic coil 30 (0.56 mm-diameter enamelled copper wire, 150 turns on a 60 mm×50 mm×15 mm 3D printed mandrel) was built to generate an approximately homogeneous magnetic field above the coil 30 (1 mT at a distance of 30 mm). It was powered by a current amplifier 35 (A1110-05-E, HUBERT, Germany), which has a gain of 1 V to 5 A. The current amplifier allows the regulation of an electric component by current instead of voltage, which is essential for the fast and precise cool-down of the electromagnetic coil 30. Between the end of the excitation phase S100 and the start of the evaluation phase S110, a short buffer time of 3 ms was applied to avoid the interference of the magnetic signal by the coil cool-down.

The noise data of the fluxgate sensor 30 in a shielded room for the simulation in this study was provided by the manufacturer (Stefan Mayer Instruments). For spatial and angular accuracy measurements, a manual linear stage (PT1, ThorLabs, Germany) with 10 μm resolution and a manual rotation stage (XRR1, ThorLabs) with 0.1° resolution were used to accurately translate and rotate the tracker 10, respectively. The translation along x-axis was measured from 0 to 50 mm with a step of 10 mm. The translation along the $z$-axis was measured from 50 mm to 75 mm distance to the sensor origin. In the presented values, a positive z is defined as being further away from the sensor plane. The rotation about the systems x-axis was measured from 0 to 90° with a step of 30°, and the rotation around the systems y-axis was measured from −45° to 45° with a step of 15°. Each measurement was repeated 10 times independently. Mean values and standard deviations were calculated and compared to the ground truth difference of two respective positions. The statistical analysis was performed in MATLAB.

Every tracker 10 can have a unique frequency response due to inaccuracies from manual fabrication even with identical cantilever materials, hence, individual resonance frequencies first have to be determined after fabrication. Consequently, for each tracker 10, amplitude filter parameters (S123) have to be adaptive. While for the frequency filter, a threshold of ±5\% of the excitation frequency is chosen, the damping coefficient threshold needs to be adjusted for each embedding material. The acceptable range is set between 5 s⁻¹ and 30 s⁻¹ for the hydrogel used for accuracy measurements and between 30 s⁻¹ and 70 s⁻¹ for the tracking demonstration. These ranges were found optimal for the respective materials. Factors which directly influence the damping coefficient, such as the proximity to container walls or the surface wetting of the tracker 10 from its environment 20, led to the choice of generous margins.

In order to avoid such outliers, boundary conditions within the algorithm are employed which limit the position H to the area below a sensing array within a 10 cm×10 cm×10 cm volume, the individual quaternions qᵢ to ±1 and the deflection angle θ between 0° and 20°. Starting values are randomized within the aforementioned ranges and the complete algorithm is repeated 10 times from which the best fit is selected by the highest R² value. The choice of generous parameter limits makes the algorithm very robust to fluctuations, however, special care has to be given for the amplitude filtering to supply the optimization algorithm with sufficient and correct information.

After fitting of B-field signals (S121), a further filtering step (S123) is applied to the matrix with entries A_{i, j}, termed amplitude filter. The main goal of the amplitude filter S123 is to detect physically unreasonable as well as strongly distorted results from the previous fitting procedure S121 and to adjust the amplitude accordingly. It is crucial since amplitude distortions or outliers directly influence the efficiency and quality of the later optimization S124. Here, three main filters are applied: a frequency filter, a damping coefficient filter and a R² filter from which a weighting matrix W is derived. Outliers can be automatically detected by using thresholds around a physically reasonable value, such as the excitation frequency. Since the damping coefficient is dependent on the embedding material of the tracker, the threshold values have to be adjusted accordingly. For R² a more generous cut-off value can be chosen due to redundancy from the physical frequency and damping filters. If any value lies outside the mentioned thresholds, its corresponding amplitude A=0 and R²=1, instead of completely removing them from the evaluation. This step increases the amount of information passed on to the later optimization S124, since no detectable oscillation implies that the amplitude is in close proximity to zero. Remaining values within thresholds are simply passed into the amplitude matrix and R² matrix, and both matrices are used for the final localization S124.

The wireless tracker 10 comprises two main parts: A screw-shaped housing 200 and a mechanically resonant structure with an attached micro-magnet. The housing 200 was designed with a computer-aided design (CAD) software (Inventor Professional 2021, Autodesk, US) and printed using a stereolithography 3D printer (3L, Formlabs, US) with 50 μm resolution and a translucent resin (Clear V4, Formlabs). Inside the housing 200, a cavity 210 was designed to allow the attachment of the cantilever and sufficient space for bending. The oscillation frequency is tunable by choice of cantilever 230 dimensions and material to achieve frequencies below within the range of 50 Hz to 1000 Hz. In this work, a 3.5 mm×0.5 mm×30 μm stripe (C1095 spring steel, Precision Brand, US) was cut by laser (MPS Advanced, Coherent, US) to form the cantilever 230, and two 1 mm×0.5 mm (diameter×length) cylindrical NdFeB magnets 220 (N52, Guys Magnets, UK) with axial magnetization were attached to the end of the cantilever 230 using cyanoacrylate adhesive (Loctite 401, Henkel, Germany).

The total theoretical magnetic moment amounts to 0.89 mAm². The cantilever 230 with fixed magnets 220 was inserted into the housing cavity 210 and closed with a 3D-printed cover. In total, the size of the screw-shaped prototype tracker 10 is 3 mm×7 mm (diameter×length).

The total theoretical magnetic moment amounts to 0.89 mAm². To avoid detachment of parts, cyanoacrylate adhesive was used. The cantilever 230 with the magnets 220 was manually inserted into the cavity 210 with a volume of 10 mm³ and closed with a fitting printed piece. In total, the size of the screw-shaped prototype tracker is 3 mm×6.5 mm. Many materials are suitable for the cantilever 230, for example spring steel, Nitinol, or biaxial polyethylene terephthalate (PET). The resonance frequency increases with increasing elastic modulus of the material, however thickness and width of the cantilever 230 are parameters to consider.

Embedding material. In order to mimic the viscoelastic properties of biological tissues, gelatine-agarose mixtures were used in this study as tissue phantoms to mimic the brain tissues, as previously reported. Hydrogel with 6 wt.-% gelatin (type A powder from porcine skin, Sigma-Aldrich, Germany) and 3 wt.-% agarose (Sigma-Aldrich) was used. Both components were stirred in double-distilled water at 80 °C for 30 min, filled in a plastic container and cooled to 22 °C for at least 4 h before use. Rectangular containers of size 50 mm×60 mm×15 mm and cylindrical containers of size 30 mm×90 mm were used. To demonstrate the propulsion of the millirobot, hydrogel with 3 wt.-% gelatin and 0.2 wt.-% agarose was used. All experiments were performed at room temperature (22 °C).

Simulation. To simulate the B-field signal of the SMOL device after the excitation, a damped harmonic oscillator model with an initial angular deflection of θ=12° was used. The corresponding differential equation was solved for θ using Simulink (MATLAB), which was applied as a time-varying input parameter for the oscillation. First, system dependent variables such as cantilever length, magnetic moment, position H and orientation T were defined and the movement of the magnetic moment in space was calculated by Eqs. 1 to 4. An accurate representation of the real measurement was achieved by calculating the time-dependent Eq. 1 from the cantilever movement at identical positions of the sensors 40 and adding recorded noise (FIG. 5C) to the signal B. Since the magnetic noise is strongly direction dependent, three different noise signals (one in each direction) were applied to the respective B-field signals, and additionally, a randomized phase shift of the added noise is introduced to reflect the arbitrary noise phase in the real measurements. Analogue-to-digital conversion is simulated as well by subdividing the data into bit-dependent increments according to the DAQ properties. Further data evaluation and localization optimization S120 were performed based on the settings of the real measurements. The numerical simulation was implemented by a customized code in MATLAB.

Mechanical excitation. As shown in FIG. 1B, the tracker 10 can also be excited by a mechanical force F, which is transmitted from the actuator 31 over a connecting piece 32 to the embedding material 20 of the tracker 10. If the actuator 31 and the connecting piece 32 are non-magnetic, the sensor 40 can record (S110) the magnetic signal B during the excitation S100 as shown in FIGS. 4A and 4B, without an additional stop (S105 in FIGS. 4C and 4D). This enables a more frequent determination of position H and orientation T of the tracker 10.

Simultaneous excitation and localization. A simultaneous excitation and localization can be performed when the excitation field does not interfere with the sensors 40.

A first method and a second method enable the simultaneous excitation and localization for magnetic excitation fields.

The first method uses an accurately known, pre-defined excitation field. The value of the pre-defined excitation field can be digitally or analogously subtracted from signal data of the sensor 40, assuming no sensor saturation to produce a remaining signal. The remaining signal is the signal of the tracking device 10. This first method means that the excitation can be performed continuously, and the localization can be performed when desired during the excitation.

The second method uses a second excitation unit. The second excitation unit creates an opposing magnetic field at each sensor 40 in the sensing array. The resulting magnetic field from the excitation at each sensor 40 is cancelled out, resulting in no effective signal during the excitation. The result of no effective signal could be achieved, for example, by mirroring the excitation coils with opposite polarity at the opposite side of the sensor array.

The first method could be performed in addition to the second method to reduce stray fields from minor differences of the mirrored excitation unit. A prerequisite for the first method and for the second method is an independency of the excitation unit and the sensing unit since the excitation unit and the sensing unit have to be operated at the same time.

A simultaneous excitation and localization can be performed when the mechanical excitation unit does not create a magnetic field for generation of mechanical fields. A mechanical wave is capable of exciting the localization device 10, i.e., the tracker. The simultaneous sensing and localization can be performed at any time during the excitation.

Multiple devices tracking. The device 10 operates at a resonance frequency which is defined and tunable over e.g., the dimensions and material of the cantilever 230. The unique frequency response of the devices 10 is used to distinguish and localize the multitude of the devices 10. The excitation S100, sensing S110 and determination S120 of the position H and orientation T of the devices 10 can be performed simultaneously or sequentially. Multiple ones of the devices 10 can be used to track relative motion of the devices 10, for example for tracking a shape or deformation of a medical instrument or a soft robot.

Array of fluxgate sensors. The magnetic field B is sensed S110 by using a multitude of sensors 40, as in an array of fluxgate magnetometers, to obtain more spatial information about the magnetic field B from the sensing S110. The individual ones of the sensors 40 withing the sensor array are spatially arranged in at least one dimension, preferably in two dimensions and the array measures the magnetic field B in at least one direction, preferably in two orthogonal directions, more preferably in three orthogonal directions.

Tumor localization. Using medical imaging, e.g., MRI, computed tomography CT, the 3D location and the shape of a tumor can be determined and reconstructed in 3D. One or more of the tracking devices 10 is injected into the tissue under the guidance of ultrasound imaging. The tracking device 10 can be placed inside or outside the tumor, preferably on the boundary of the tumor. The location H and orientation T of the device 10 are imaged by non-magnetic medical imaging, e.g., CT, and the spatial relative position and orientation of the tumor to the trackers are calculated based on these non-magnetic medical images. In a situation, where large medical imaging is not possible, e.g., in an operation room, the trackers are localized with the method set out in this document, and the real-time position and orientation and shape of the tumor are calculated based on the localization information of the device 10. Such information is displayed as overlaid images onto the patient body for the surgical scene, e.g., via a screen, a projector, or a pair of augmented reality glasses. The method can locate substantially accurately the tumor's real-time position, regardless of soft tissue deformation, thus the method offers an accurate way to remove the tumor by surgery. The embedded devices 10 are removed from the body together with the tumor after the procedure. The method is generally able to track also other important anatomies, such as blood vessels, blood clots, polyps, nerves. The procedures are not limited to tumor surgery, but also apply to other medical procedures where localization is needed, such as radiotherapy, targeted chemotherapy, selected embolization, etc.

Actuation system. For the actuation of the millirobot R, an external rotating magnetic field was used. A cubic NdFeB magnet (side length 50.8 mm, N40, Supermagnete, Germany) was mechanically fixed to a stepper motor (23HS30-2804S, Stepperonline, US) with the magnetic axis perpendicular to the rotational axis of the motor. The assembly was placed on a rotational stage (GFV5G50, Orientalmotor, Japan) to steer the rotational axis of the magnet in 2D plane. The propulsion of the millirobot R was restricted to a planar motion in the gel in between a PMMA plate and the bottom of the container with a gap of 6 mm and the propulsion path was pre-defined in the gel. The speed of 0.25 Hz and the direction of the rotating magnetic field was controlled by an Arduino board (Ardunio Uno Rev3 SMD, Arduino), and the direction of the rotational axis was steered manually. The propulsion was paused for 8 times for the localization H, T. In each pause, the sample box (with the robot R embedded statically inside) was removed from the actuation set-up and put in the localization set-up. After that, the propulsion was continued in the actuation set-up. A LED light source was applied for illumination, and a camera system (Canon EOS RP RF 24-105 mm F4 with a RF35 mm F1.8 lens at 25 fps, Canon, Japan) was used to image the propulsion from the top view. 8 videos were linked and analyzed by a customized code (MATLAB) to recognize the robot center-point and orientation (HT1 to HT9 in FIG. 14) in each frame and draw the trajectory to overlay on the original videos.

Complementary Ultrasound imaging. Porcine brains were obtained from a local butcher, transported on ice, and stored in the fridge at 4 °C. All experiments were done within 12 h after the sacrifice of the animal. The sample was hydrated with phosphate-buffered saline solution (PBS, Sigma-Aldrich) and put in a 60 mm×50 mm×25 mm container for measurements at room temperature. For US imaging, a handheld US machine (iQ+, Butterfly Network, US) was used with the setting “MSK-Soft Tissue” at a frequency of 1-10 MHz, with a thermal index for soft tissue (TIS) of 0.01 and a mechanical index (MI) of 0.28. The US probe was set in contact with the porcine brain using US contacting gel (Aquasonic 100, Parker Laboratories, US).

Mechanical property testing of viscoelastic media, such as biological soft tissues, is crucial for the understanding and characterization of the complex, multi-composite, soft materials. In medicine, elastic properties are examined by palpation, a process of measuring the elastic response of tissue by direct contact. Such mechanical tests are carried out by quasi-static or dynamic indentation tests with an external load, rheometry or mechanical wave propagation. Results from such tests are then used to model or estimate material behaviors of viscoelastic tissues. However, the ex vivo tests may not represent the in vivo physiological conditions and the material's surface structures, e.g., the skin, which usually exhibit strongly different properties as the bulk material. The brain, as another example, is surrounded by a thin protective layer, the pia mater, which has a high elastic modulus in comparison to the brain bulk material. This discrepancy influences the bulk material testing with the force that is applied externally and does not yield the true bulk properties.

A more general approach for bulk material properties is obtained from mechanical wave propagation in combination with imaging. One such approach for mechano-sensing in biological tissues is magnetic resonance elastography (MRE) which can image elastic properties directly by assessing the propagation of mechanical waves. However, very large and complex magnetic MR machinery is required, which makes it unsuitable for the long-term measurements and monitoring of elasticity developments. Mechanical resonant structures have been proposed for materials sensing, such as magnetic MEMS devices for the pressure sensing and tethered piezo-driven cantilevers for viscoelastic characterization.

To determine S115 wirelessly the local viscoelastic properties of soft biological materials, the localization device 10 can be used. The localization 10 device can be directly embedded into the embedding material 20. Over its the mechanical resonance frequency and damping response of the localization device 10, material properties can be determined (in step S115) wirelessly without any expensive equipment. The localization device (10) can be implemented by minimally invasive procedures and can be used for the long-term monitoring of the mechanical properties change in soft biomaterials.

An artificial hydrogel made of gelatin was prepared to mimic biological soft tissues and to validate the functionality of the localization device 10. Gelatin hydrogels of 2 wt.-\% to 4 wt.-\% (type A from porcine skin, Sigma-Aldrich) were prepared by stirring gelatin powder for 30 min at 80 °C in distilled water. The mixture was poured into a 60 mm×50 mm container and cooled to room temperature for at least 12 h before use. Dehydration was prevented by covering the container with a lid. Ex vivo tissue specimens, i.e., turkey breast, porcine liver, and brain, were obtained from the local butcher and tested within 12 h after the sacrifice of the animal. During tests at room temperature, the samples were hydrated with phosphate-buffered saline solution (PBS, Sigma-Aldrich) and put in a 60 mm×50 mm container for measurements.

The localization device 10 comprises an elastic spring element 230 with spring constant k₁ which has an internal damping of η₁. The spring element 230 is coupled to two masses: m₁ being the magnet 220 attached to the free end 235 of the spring element 230, and m₂ being an effective mass consisting of the housing 200 and the embedding material 20) The embedding material 20 can be a viscoelastic material. In one example the embedding material 20 can be modelled as Maxwell or Kelvin-Voigt linear models and is shown with dashed lines in FIG. 19.

Two localization devices 10 were manually assembled with resonant frequencies fᵣₑₛ=90.33 Hz and fᵣₑₛ=100.73 Hz using two 1 mm×0.5 mm cylindrical N52 NdFeB magnets (glued onto a steel cantilever of 20 μm thickness, 200 μm width and 2.5 mm length, all inside a 3D printed housing 200. For soft material measurements, such as the biological material, the device 10 was embedded into the sample 20 with at least 10 mm distance to any hard boundary.

A rigid boundary reference was measured by fixing the localization device 10 in an enclosure rigidly attached to a frame. Under the assumption of an ideal, damped mass-spring system with a quasi-infinite mass m₂ (see FIG. 19), the spring constant k₁ can be calculated by:

$$f = \frac{1}{2 π} \sqrt{\frac{{k}_{1}}{{m}_{1}} - {η}_{1}^{2}} \qquad ( 10 )$$

where f=fᵣₑₛ and with m₁ as the magnets 220 mass. 11=nr is the damping coefficient with rigid boundary conditions measured from the decaying magnetic field (Eq. 1). With m₁=(7.9±0.1)×10⁻⁶ kg, f_{res,1}=(90.33±0.04) Hz and ηᵣ=(4.2±0.1) s⁻¹, k₁ becomes (2.54±0.03) Nm⁻¹. The spring constant can also be determined by material and dimension considerations using the formula for a cantilever:

$$k *= \frac{3 EI}{{L}^{3}} \qquad ( 11 )$$

with E as the Youngs modulus, the area moment of inertia

$$l = \frac{3 {wh}^{3}}{12}$$

(w width, h height) and the cantilever length L. This results in an estimated spring constant k* in the range of 1.2 to 2.54 Nm⁻¹. The error margin stems from production uncertainties. k₁ lies within the estimated range of k*, validating the assumption of a simple mass-spring system.

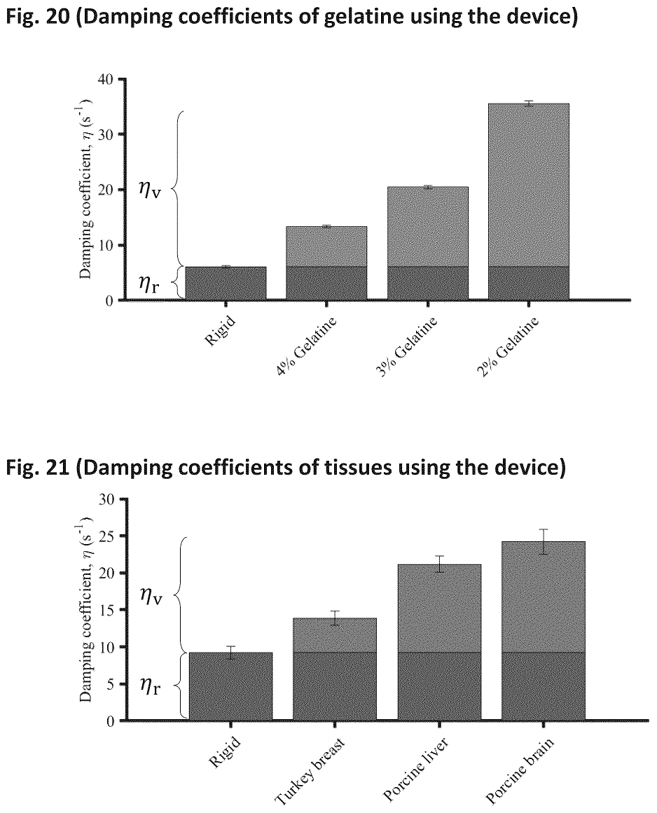

As mentioned, the internal damping η₁ of the localization device 10 can be obtained by Eq. 7. This value can be compared to systems with soft coupling of m₂ to a viscoelastic environment 20, as demonstrated for three concentrations of gelatin hydrogels in FIG. 20. Since nr is the lower limit of possible damping, the total damping of the system can be described by the sum of the rigid and viscoelastic contribution η=ηᵣ+η_{V}·η increases significantly by the insertion of the device (10) in gelatin and with the decrease of gelatin contents. This phenomenon is expected since the gelatin matrix stiffens with gelatin content due to physical bonding by molecular entanglement. The increase of gelatin stiffness with its concentration is reported in literature, which is consistent with the current observation over the damping coefficient determined by the device (10).

Furthermore, the damping behavior of various ex vivo tissues was measured and is presented in FIG. 21. The device 10 used for these experiments comprises a spring constant of k₁=(3.17±0.04) Nm⁻¹. Damping coefficients were determined between (9.2±0.9) s⁻¹ for the rigid boundary and (24.2±1.6) s⁻¹ for porcine brain. η increases significantly between turkey breast, porcine liver, and porcine brain, matching well to the decreasing stiffness of these biological tissues. While in literature the reported stiffness between the muscle fiber in the turkey breast and the brain tissue varies by two orders of magnitude, η only varies by approximately a factor of around 1.7. This is an indication of non-linear dependence between the damping coefficient and material stiffness.

Reference Numerals

| | |
|---|---|
| 5 | Apparatus |
| 10 | Localization device/Tracker |
| 20 | Soft tissue/Embedding material |
| 30 | Magnetic excitation coil |
| 31 | Mechanical excitation source |
| 32 | Connecting piece |
| 35 | Current amplifier |
| 40 | Sensor |
| 45 | Positioning stage |
| 50 | Data acquisition and computation unit |
| 200 | Housing |
| 210 | Cavity |
| 220 | Magnet |
| 225 | Dipole center |
| 230 | Oscillating Element/Cantilever |
| 235 | First end |
| 237 | Rotational axis |
| 240 | Movement path |

Reference letters

| | |
|---|---|
| F | Excitation field |
| Θ | Deflection angle |
| B | Magnetic field |
| H | Position of 10 |
| T | Orientation of 10 |
| R | Robot comprising 10 |
| P | Actuation path of R |

1. A method for determining a position and an orientation of a localization device, the localization device having a magnet attached to an oscillating element, the method comprising:
exciting the magnet using one of an external force or torque, resulting in a complex oscillatory motion of the magnet, wherein said complex motion comprises translation and rotation of the magnetic moment of the magnet about a rotational axis and the rotational axis is located at an offset distance relative to the center of the magnetic moment of the magnet;
sensing the magnetic field generated by the magnet using at least one sensor, and
determining the position and the orientation of the localization device from the sensing.

2. The method of claim 1, wherein the exciting using the one of the external force or torque is powered by one of an external magnetic excitation field, or a mechanical excitation.

3. The method of claim 2, wherein the mechanical exciting is one of a pulse and a wave oscillating in at least one of a longitudinal direction or a shear direction of the oscillating element.

4. The method of claim 1, wherein the distance between the said rotational axis and the magnetic moment of the magnet is larger than, at least 5%, preferably 25%, more preferably 100% of the largest dimension of the magnet.

5. The method of claim 1, wherein the rotational axis and the magnetic moment vector stemming from the magnet are non-parallel, and preferably substantially perpendicular.

6. The method of claim 1, wherein the oscillating element is located within a rigid housing, more preferably a sealed rigid housing with vacuum.

7. The method of claim 1, wherein at least one of the sensing or the determining of the position and orientation of the localization device is performed when at least one of the exciting of the magnet is stopped or performed continuously during the exciting of the magnet.

8. The method of claim 1, wherein the oscillating element comprises a restoring force unit at least one of a cantilever beam or a similar unit providing an elastic restoring force.

9. The method of claim 1, wherein the sensing and the determining of the position and the orientation of the device are repeated.

10. The method of claim 6, wherein the frequency of excitation of the magnet is approximately the resonance frequency of the oscillating element.

11. The method of claim 1, wherein the determining of the position and the orientation of the localization device comprises using a physical model of the complex oscillatory motion in a time domain.

12. The method of claim 1, wherein the sensing of the magnetic field generated by the magnet is followed by determining local viscoelastic properties of material.

13. The method of claim 11, further comprising using the determined local viscoelastic properties of the material to determine the position and orientation of the device.

14. An apparatus for determining a position and an orientation of a localization device, the localization device having a housing within which a magnet attached to an oscillating element is located, comprising:
an excitation unit for exciting the magnet using one of an external force or torque resulting in a complex oscillatory motion of the magnet, wherein said complex motion comprises translation and rotation of the magnetic moment of the said magnet about a rotational axis located at an offset distance relative to the center of the magnetic moment of the magnet;
a localization device having a housing within which the magnet is attached to the oscillating element; and
a data acquisition unit for sensing a magnetic field B generated by the magnet using a sensor.

15. The apparatus of claim 14, wherein the magnet comprises a permanent magnet made of a magnetic material, preferably a ferromagnetic material.

16. The apparatus of claim 14, wherein the oscillating element comprises a restoring force unit, preferably a cantilever.

17. Use of the apparatus of claim 14, to localize a medical implant in an animal or a human body, chosen from one or more of a catheter, a stent, a guidewire, an endoscope, a capsule endoscope, a drug delivery device or a small-scale robot, or to localize an anatomy, chosen from one or more of a tumor, blood vessel, blood clot, polyp, or nerve.
